SEQUENCE LISTING (6 sequences)

SEQ ID NO: 1 - DNA, 8519 bp, Homo sapiens
  source 1..8519: mol_type = genomic DNA
atggccgcgcacaggccggtggaatgggtccaggccgtggtcagccgcttcgacgagcagcttccaataaaaacaggacagcagaacacacataccaaagtcagtactgagcacaacaaggaatgtctaatcaatatttccaaatacaagttttctttggttataagcggcctcactactattttaaagaatgttaacaatatgagaatatttggagaagctgctgaaaaaaatttatatctctctcagttgattatattggatacactggaaaaatgtcttgctgggcaaccaaaggacacaatgagattagatgaaacgatgctggtcaaacagttgctgccagaaatctgccattttcttcacacctgtcgtgaaggaaaccagcatgcagctgaacttcggaattctgcctctggggttttattttctctcagctgcaacaacttcaatgcagtctttagtcgcatttctaccaggttacaggaattaactgtttgttcagaagacaatgttgatgttcatgatatagaattgttacagtatatcaatgtggattgtgcaaaattaaaacgactcctgaaggaaacagcatttaaatttaaagccctaaagaaggttgcgcagttagcagttataaatagcctggaaaaggcattttggaactgggtagaaaattatccagatgaatttacaaaactgtaccagatcccacagactgatatggctgaatgtgcagaaaagctatttgacttggtggatggttttgctgaaagcaccaaacgtaaagcagcagtttggccactacaaatcattctccttatcttgtgtccagaaataatccaggatatatccaaagacgtggttgatgaaaacaacatgaataagaagttatttctggacagtctacgaaaagctcttgctggccatggaggaagtaggcagctgacagaaagtgctgcaattgcctgtgtcaaactgtgtaaagcaagtacttacatcaattgggaagataactctgtcattttcctacttgttcagtccatggtggttgatcttaagaacctgctttttaatccaagtaagccattctcaagaggcagtcagcctgcagatgtggatctaatgattgactgccttgtttcttgctttcgtataagccctcacaacaaccaacactttaagatctgcctggctcagaattcaccttctacatttcactatgtgctggtaaattcactccatcgaatcatcaccaattccgcattggattggtggcctaagattgatgctgtgtattgtcactcggttgaacttcgaaatatgtttggtgaaacacttcataaagcagtgcaaggttgtggagcacacccagcaatacgaatggcaccgagtcttacatttaaagaaaaagtaacaagccttaaatttaaagaaaaacctacagacctggagacaagaagctataagtatcttctcttgtccatggtgaaactaattcatgcagatccaaagctcttgctttgtaatccaagaaaacaggggcccgaaacccaaggcagtacagcagaattaattacagggctcgtccaactggtccctcagtcacacatgccagagattgctcaggaagcaatggaggctctgctggttcttcatcagttagatagcattgatttgtggaatcctgatgctcctgtagaaacattttgggagattagctcacaaatgcttttttacatctgcaagaaattaactagtcatcaaatgcttagtagcacagaaattctcaagtggttgcgggaaatattgatctgcaggaataaatttcttcttaaaaataagcaggcagatagaagttcctgtcactttctccttttttacggggtaggatgtgatattccttctagtggaaataccagtcaaatgtccatggatcatgaagaattactacgtactcctggagcctctctccggaagggaaaagggaactcctctatggatagtgcagcaggatgcagcggaacccccccgatttgccgacaagcccagaccaaactagaagtggccctgtacatgtttctgtggaaccctgacactgaagctgttctggttgccatgtcctgtttccgccacctctgtgaggaagcagatatccggtgtggggtggatgaagtgtcagtgcataacctcttgcccaactataacacattcatggagtttgcctctgtcagcaatatgatgtcaacaggaagagcagcacttcagaaaagagtgatggcactgctgaggcgcattgagcatcccactgcaggaaacactgaggcttgggaagatacacatgcaaaatgggaacaagcaacaaagctaatccttaactatccaaaagccaaaatggaagatggccaggctgctgaaagccttcacaagaccattgttaagaggcgaatgtcccatgtgagtggaggaggatccatagatttgtctgacacagactccctacaggaatggatcaacatgactggcttcctttgtgcccttgggggagtgtgcctccagcagagaagcaattctggcctggcaacctatagcccacccatgggtccagtcagtgaacgtaagggttctatgatttcagtgatgtcttcagagggaaacgcagatacacctgtcagcaaatttatggatcggctgttgtccttaatggtgtgtaaccatgagaaagtgggacttcaaatacggaccaatgttaaggatctggtgggtctagaattgagtcctgctctgtatccaatgctatttaacaaattgaagaataccatcagcaagttttttgactcccaaggacaggttttattgactgataccaatactcaatttgtagaacaaaccatagctataatgaagaacttgctagataatcatactgaaggcagctctgaacatctagggcaagctagcattgaaacaatgatgttaaatctggtcaggtatgttcgtgtgcttgggaatatggtccatgcaattcaaataaaaacgaaactgtgtcaattagttgaagtaatgatggcaaggagagatgacctctcattttgccaagagatgaaatttaggaataagatggtagaatacctgacagactgggttatgggaacatcaaaccaagcagcagatgatgatgtaaaatgtcttacaagagatttggaccaggcaagcatggaagcagtagtttcacttctagctggtctccctctgcagcctgaagaaggagatggtgtggaattgatggaagccaaatcacagttatttcttaaatacttcacattatttatgaaccttttgaatgactgcagtgaagttgaagatgaaagtgcgcaaacaggtggcaggaaacgtggcatgtctcggaggctggcatcactgaggcactgtacggtccttgcaatgtcaaacttactcaatgccaacgtagacagtggtctcatgcactccataggcttaggttaccacaaggatctccagacaagagctacatttatggaagttctgacaaaaatccttcaacaaggcacagaatttgacacacttgcagaaacagtattggctgatcggtttgagagattggtggaactggtcacaatgatgggtgatcaaggagaactccctatagcgatggctctggccaatgtggttccttgttctcagtgggatgaactagctcgagttctggttactctgtttgattctcggcatttactctaccaactgctctggaacatgttttctaaagaagtagaattggcagactccatgcagactctcttccgaggcaacagcttggccagtaaaataatgacattctgtttcaaggtatatggtgctacctatctacaaaaactcctggatcctttattacgaattgtgatcacatcctctgattggcaacatgttagctttgaagtggatcctaccaggttagaaccatcagagagccttgaggaaaaccagcggaacctccttcagatgactgaaaagttcttccatgccatcatcagttcctcctcagaattcccccctcaacttcgaagtgtgtgccactgtttataccaggcaacttgccactccctactgaataaagctacagtaaaagaaaaaaaggaaaacaaaaaatcagtggttagccagcgtttccctcagaacagcatcggtgcagtaggaagtgccatgttcctcagatttatcaatcctgccattgtctcaccgtatgaagcagggattttagataaaaagccaccacctagaatcgaaaggggcttgaagttaatgtcaaagatacttcagagtattgccaatcatgttctcttcacaaaagaagaacatatgcggcctttcaatgattttgtgaaaagcaactttgatgcagcacgcaggtttttccttgatatagcatctgattgtcctacaagtgatgcagtaaatcatagtctttccttcataagtgacggcaatgtgcttgctttacatcgtctactctggaacaatcaggagaaaattgggcagtatctttccagcaacagggatcataaagctgttggaagacgaccttttgataagatggcaacacttcttgcatacctgggtcctccagagcacaaacctgtggcagatacacactggtccagccttaaccttaccagttcaaagtttgaggaatttatgactaggcatcaggtacatgaaaaagaagaattcaaggctttgaaaacgttaagtattttctaccaagctgggacttccaaagctgggaatcctattttttattatgttgcacggaggttcaaaactggtcaaatcaatggtgatttgctgatataccatgtcttactgactttaaagccatattatgcaaagccatatgaaattgtagtggaccttacccataccgggcctagcaatcgctttaaaacagactttctctctaagtggtttgttgtttttcctggctttgcttacgacaacgtctccgcagtctatatctataactgtaactcctgggtcagggagtacaccaagtatcatgagcggctgctgactggcctcaaaggtagcaaaaggcttgttttcatagactgtcctgggaaactggctgagcacatagagcatgaacaacagaaactacctgctgccaccttggctttagaagaggacctgaaggtattccacaatgctctcaagctagctcacaaagacaccaaagtttctattaaagttggttctactgctgtccaagtaacttcagcagagcgaacaaaagtcctagggcaatcagtctttctaaatgacatttattatgcttcggaaattgaagaaatctgcctagtagatgagaaccagttcaccttaaccattgcaaaccagggcacgccgctcaccttcatgcaccaggagtgtgaagccattgtccagtctatcattcatatccggacccgctgggaactgtcacagcccgactctatcccccaacacaccaagattcggccaaaagatgtccctgggacactgctcaatatcgcattacttaatttaggcagttctgacccgagtttacggtcagctgcctataatcttctgtgtgccttaacttgtacctttaatttaaaaatcgagggccagttactagagacatcaggtttatgtatccctgccaacaacaccctctttattgtctctattagtaagacactggcagccaatgagccacacctcacgttagaatttttggaagagtgtatttctggatttagcaaatctagtattgaattgaaacacctttgtttggaatacatgactccatggctgtcaaatctagttcgtttttgcaagcataatgatgatgccaaacgacaaagagttactgctattcttgacaagctgataacaatgaccatcaatgaaaaacagatgtacccatctattcaagcaaaaatatggggaagccttgggcagattacagatctgcttgatgttgtactagacagtttcatcaaaaccagtgcaacaggtggcttgggatcaataaaagctgaggtgatggcagatactgctgtagctttggcttctggaaatgtgaaattggtttcaagcaaggttattggaaggatgtgcaaaataattgacaagacatgcttatctccaactcctactttagaacaacatcttatgtgggatgatattgctattttagcacgctacatgctgatgctgtccttcaacaattcccttgatgtggcagctcatcttccctacctcttccacgttgttactttcttagtagccacaggtccgctctcccttagagcttccacacatggactggtcattaatatcattcactctctgtgtacttgttcacagcttcattttagtgaagagaccaagcaagttttgagactcagtctgacagagttctcattacccaaattttacttgctgtttggcattagcaaagtcaagtcagctgctgtcattgccttccgttccagttaccgggacaggtcattctctcctggctcctatgagagagagacttttgctttgacatccttggaaacagtcacagaagctttgttggagatcatggaggcatgcatgagagatattccaacgtgcaagtggctggaccagtggacagaactagctcaaagatttgcattccaatataatccatccctgcaaccaagagctcttgttgtctttgggtgtattagcaaacgagtgtctcatgggcagataaagcagataatccgtattcttagcaaggcacttgagagttgcttaaaaggacctgacacttacaacagtcaagttctgatagaagctacagtaatagcactaaccaaattacagccacttcttaataaggactcgcctctgcacaaagccctcttttgggtagctgtggctgtgctgcagcttgatgaggtcaacttgtattcagcaggtaccgcacttcttgaacaaaacctgcatactttagatagtctccgtatattcaatgacaagagtccagaggaagtatttatggcaatccggaatcctctggagtggcactgcaagcaaatggatcattttgttggactcaatttcaactctaactttaactttgcattggttggacaccttttaaaagggtacaggcatccttcacctgctattgttgcaagaacagtcagaattttacatacactactaactctggttaacaaacacagaaattgtgacaaatttgaagtgaatacacagagcgtggcctacttagcagctttacttacagtgtctgaagaagttcgaagtcgctgcagcctaaaacatagaaagtcacttcttcttactgatatttcaatggaaaatgttcctatggatacatatcccattcatcatggtgacccttcctataggacactaaaggagactcagccatggtcctctcccaaaggttctgaaggataccttgcagccacctatccaactgtcggccagaccagtccccgagccaggaaatccatgagcctggacatggggcaaccttctcaggccaacactaagaagttgcttggaacaaggaaaagttttgatcacttgatatcagacacaaaggctcctaaaaggcaagaaatggaatcagggatcacaacaccccccaaaatgaggagagtagcagaaactgattatgaaatggaaactcagaggatttcctcatcacaacagcacccacatttacgtaaagtttcagtgtctgaatcaaatgttctcttggatgaagaagtacttactgatccgaagatccaggcgctgcttcttactgttctagctacactggtaaaatataccacagatgagtttgatcaacgaattctttatgaatacttagcagaggccagtgttgtgtttcccaaagtctttcctgttgtgcataatttgttggactctaagatcaacaccctgttatcattgtgccaagatccaaatttgttaaatccaatccatggaattgtgcagagtgtggtgtaccatgaagaatccccaccacaataccaaacatcttacctgcaaagttttggttttaatggcttgtggcggtttgcaggacgttttcaaagcaaacacaaattccagactatgctgagcttattgttaagtttcttgatgccttgattgacacgtacctgcctggaattgatgaagaaaccagtgaagaatccctcctgactcccacatctccttaccctcctgcactgcagagccagcttagtatcactgccaaccttaacctttctaattccatgacctcacttgcaacttcccagcattccccaggaatcgacaaggagaacgttgaactctcccctaccactggccactgtaacagtggacgaactcgccacggatccgcaagccaagtgcagaagcaaagaagcgctggcagtttcaaacgtaatagcattaagaagatcgtgtga

SEQ ID NO: 2 - DNA, 8517 bp, synthetic construct
  source 1..8517: mol_type = other DNA
atggcagctcacaggcccgtagaatgggttcaagcggtagtaagccgatttgacgaacagctgccgattaaaacaggtcaacaaaacacgcacaccaaggttagcactgagcacaacaaggagtgcctgatcaacatttccaaatataaattttccctggtgatctccggtctgaccaccatcctcaaaaacgtgaacaatatgcgtatttttggagaagctgcagagaaaaatctgtacctgtctcaattgattatactggatactttagagaaatgcctggcgggccaaccaaaggatacgatgcgcctggacgagaccatgctggtgaagcaattattgccggagatctgccactttctgcacacctgccgtgaaggaaatcagcacgccgccgaattacgcaattctgcgtccggcgtcttgtttagcctgagttgcaacaacttcaatgcagtctttagccgtatctccaccaggttgcaggagctgaccgtgtgtagcgaggacaacgtggacgttcatgatatcgagctgctgcaatatattaacgttgattgtgcaaaactgaaacgtctgctcaaagaaaccgccttcaagtttaaggcgttaaagaaagttgcgcaactagcggttatcaattctctcgagaaagcgttctggaattgggttgaaaattatccggatgagtttaccaagctgtatcagatcccgcagaccgatatggctgagtgcgcggaaaagctgtttgatttggttgatggtttcgcagaatctacaaaacgcaaggccgctgtgtggccgctccaaattatcctgctgattctgtgtccggaaatcatccaagatatctcaaaggatgttgtggatgaaaacaatatgaataagaaactgttcctagactctttgcgaaaggcgttagcgggtcatggtggtagccgtcagctgaccgagagcgcggcgatagcgtgtgtaaaactgtgcaaagcttccacctacattaattgggaagataatagtgtgatcttcctgctggtccagtcgatggttgttgacctgaagaacctgctgtttaacccgtctaaaccgtttagccgtggtagccaaccggcggatgtggacctgatgattgactgtctggtgtcttgcttccgcatctcaccgcataacaaccagcattttaaaatctgcctcgctcagaatagcccttctaccttccattatgttctggtgaactcactgcaccgtatcatcacgaattctgccctggattggtggcctaaaattgatgcggtttactgccacagcgtggaattgagaaacatgtttggtgaaacgctgcacaaggcggtgcagggttgtggtgcacacccggctatccgcatggcaccgtcgttgacgtttaaggagaaggtgaccagtcttaagttcaaggagaaaccgaccgacctcgaaacccgttcctacaaatatctgctgctgagcatggttaaactgatccatgcggaccctaaattgctcctctgcaatccgagaaagcagggcccagaaacccaaggttctacggcggaactgatcaccggtttggtgcaactcgtcccacagtcccatatgccggaaattgcccaagaggcaatggaagcgctcctggtgctgcaccagttggactcaatcgacctttggaatccggatgcgccagttgagaccttttgggagatttcttcacaaatgcttttttacatctgcaagaaactgaccagccaccagatgctgtctagcaccgaaattctgaagtggctgcgcgaaattctcatttgtcgtaataagttcttgttaaagaacaagcaagcggatcgttccagctgccactttctgttattctacggtgttggctgcgacataccgagctctggcaatacctcccagatgtcgatggatcatgaggagctgctccgcaccccaggcgcatccctacgcaaaggtaaaggtaactcaagcatggatagcgcagctggctgctccgggaccccgccgatctgccgtcaggctcaaactaagttggaggttgcactttacatgttcctgtggaacccggacaccgaagcggtcctggttgctatgtcgtgctttcgtcacctgtgcgaggaagcggacattcgctgcggtgttgatgaagttagcgtccataatctgctgccgaattacaatacctttatggaatttgcgtccgtgtccaatatgatgagtacgggccgcgcagcgctgcagaagcgtgttatggcgcttctgagaaggatcgagcacccgactgcaggcaacaccgaggcgtgggaggacacgcacgctaaatgggaacaagcaaccaagctgatcctcaactacccgaaagcgaaaatggaggacggccaagcagctgagtccttgcacaaaacaattgtaaaacgtcgcatgagccatgtgagcggcggcggcagcatcgacttatccgacacggacagcttgcaagagtggattaatatgactggctttctttgcgctctgggtggtgtgtgcctgcagcagcgttccaattccggcttggcaacgtacagcccgccgatgggtccagttagcgagcgtaaaggcagcatgatctccgtgatgagctcagagggtaatgcggatacgccggtttcgaagtttatggatcgtttactcagcttgatggtgtgtaatcacgaaaaagtgggcctgcaaattcgtacgaacgttaaggaccttgtgggcctggaactgtcccctgctttgtacccgatgctgttcaacaagcttaaaaacaccatctctaaattcttcgatagccagggccaagttttgctgacagacaccaatacgcagtttgtagagcagacgattgcgattatgaaaaacctgctggacaaccacaccgaggggagctccgagcacttgggtcaggcgtctatcgagacgatgatgctcaacctggttcgttatgtgcgtgttctgggcaatatggtacatgcgatccaaattaaaaccaagctatgccaactggttgaagtgatgatggcgcgtcgcgatgatctgtctttctgccaagaaatgaaatttcgcaacaaaatggtcgagtacttgacggactgggtgatgggaacgtcgaatcaggctgccgacgacgacgtgaagtgcctgacgcgcgatctggaccaggccagcatggaggcggtggtgtccttgcttgcggggctgccattgcagccggaagagggcgacggcgttgagttgatggaagcgaaatcccaactgttcttaaaatactttaccctgttcatgaacctgctcaacgactgtagcgaagttgaggatgaaagcgcccagactggcggccgtaaacgaggtatgagtcgtcgtctagcgtctctgcgtcactgcaccgttctggcgatgtcgaatctgctgaatgcaaacgtagactccggcctgatgcattctattggtttgggctaccataaagacttacaaacgcgtgcgacctttatggaggtgctgaccaagatcctgcagcaaggcaccgagttcgacacgcttgctgaaaccgttctggccgatcgtttcgaacgtctggttgagttggtaacgatgatgggtgatcagggtgaactgccgatcgcaatggcattagccaacgtggtgccgtgttcgcagtgggatgagttagcgcgtgttcttgttaccctgttcgatagccgccatttattatatcaactgttgtggaacatgtttagcaaggaagtagaattggcggatagcatgcagaccctgttcagaggaaactcgttggcctcgaagattatgaccttttgcttcaaagtttatggtgctacctacttgcaaaaactcctagacccgctgttaagaatcgtgattacctcatccgattggcagcatgtttcttttgaagtcgatccgactcgcctggagccgtccgagagcttggaggagaaccagagaaacctgctgcagatgaccgagaagttctttcacgcgattatcagtagctcctccgaatttccgccgcagctccggtccgtttgccactgcctgtaccaagctacatgccatagcttactcaataaagctaccgtgaaagaaaagaaagagaataaaaagtcagtggtcagccagcgctttccgcagaacagcatcggtgcagtaggctctgccatgtttctgcgattcatcaatccggccatcgtgagcccgtatgaagcgggtattctggacaagaagccgccgccgcgtattgaacgtggtctgaaattgatgagcaagatcctccaaagcattgcgaatcatgttctcttcaccaaggaagagcacatgcgtccgtttaacgattttgtgaaatccaattttgatgctgcacgtcgttttttcctggatatcgcgagcgactgcccaacgagcgacgcagtgaaccatagtctgtctttcatctctgacggtaacgtgctggccctccacagattactgtggaataaccaagaaaagatcggccaatacctctccagcaaccgtgaccacaaagcggtaggtagacgtccattcgacaaaatggcgacgctgttggcatatctaggcccaccggagcacaaaccggttgcggacacccattggagcagcctgaacttgactagctccaaattcgaagagtttatgacccgtcatcaggtgcacgaaaaggaagagttcaaggcgctgaaaaccctctctattttctatcaggccggcaccagcaaagcgggtaacccgatcttctactacgtggcgcgcagattcaaaaccggccagatcaatggtgatctgctgatctaccatgtgcttctgactctgaaaccgtactatgccaagccgtatgaaattgttgtggacttgacccacactggtccgagtaaccgcttcaaaaccgatttcctgagcaaatggtttgttgtgtttccgggttttgcgtatgataacgtctcagctgtttatatctataattgtaacagctgggttcgtgaatacaccaagtaccatgaacgtctgctgacgggtctgaagggtagcaagcgcctggtgttcattgattgtccgggtaaattggccgagcacattgaacacgaacagcaaaaactgccggcggctaccctggcacttgaggaggatctgaaggtgttccacaacgctctgaagctggcccataaagatacgaaagtgtccattaaggttggttctacagcggtccaggtaacctcggcagaacgtaccaaagtgcttggtcagtctgtctttctaaatgacatctactacgctagcgagatcgaagaaatctgcctggttgacgagaaccagtttacgcttaccatcgctaatcagggtacgccactgacgttcatgcaccaggagtgcgaagctattgtccagtccatcattcatattcgtacccgttgggagctgtcccaaccggatagtatcccgcaacacaccaagattcgaccgaaagacgtgcccggtaccctcctcaacattgcgttactcaatttgggtagtagcgacccgtcccttcgttctgcggcctataacctgctttgcgctctgacgtgcacgttcaaccttaagatcgagggccaactgctggaaacgtccggtttatgtattccggcgaataacacgctgttcatcgtgtccataagcaagaccctcgcggcaaatgaaccgcatctgacgctggaattcctggaagagtgcattagtggtttcagcaagagcagcatcgaattgaagcacctgtgtctggaatatatgaccccgtggctgtctaacctggttcgtttctgtaaacataatgatgatgcgaaacgccagcgtgtgaccgccatcctggacaaactgatcaccatgaccattaacgagaagcagatgtatcctagtatccaggctaagatttggggttctttaggtcaaatcaccgatttgctggacgtcgttctggatagcttcattaaaaccagcgctaccggtggtctgggtagcatcaaagcggaagtgatggccgatacggcagtggctttagcgagcggtaacgtgaagttggttagttcaaaagttattggtcgtatgtgtaaaattatcgacaagacctgtcttagtccgactccgaccctggaacaacatctgatgtgggatgatattgcgattcttgctcgatatatgcttatgctgtcattcaacaactctttggatgtcgcggcgcacctgccatacttattccacgtggtgaccttcctggttgctaccggaccgctgagcctccgtgcaagcactcatggccttgtcatcaacatcatccacagcctgtgcacgtgcagtcagctgcacttcagcgaggagaccaagcaggttctccgtctctccctgaccgagttctcgctgccgaaattttatttactcttcggcatcagcaaggttaagagcgccgcggttatcgcgtttcgcagcagctaccgcgaccgttccttcagcccgggctcttacgaacgcgaaacattcgcgctcacctcgctggaaactgtcaccgaagcgctgcttgagatcatggaagcctgcatgcgtgatatcccgacctgcaagtggctggaccaatggaccgagctggcccaacgttttgcgttccagtataacccgagcctacaaccgcgtgcgctcgttgtgttcggctgcataagcaaacgtgtttcccatggtcaaattaagcagatcattcgtatcctctcgaaagccttggagagctgtctgaagggtcccgatacctacaattctcaggttctgatcgaggcaactgtgatcgctttaaccaaactgcaaccgttgctgaataaagatagcccgctgcacaaagctctgttttgggttgcagtggccgtcctgcagctggacgaagtgaacctgtacagcgcgggtaccgccctgctggagcaaaatttgcacaccctggacagcttgcgcatctttaatgacaaatccccagaagaggtttttatggccatccgcaacccgctggagtggcattgtaaacaaatggatcacttcgtcggcttgaactttaatagcaactttaacttcgcgctcgtgggccatctgttaaagggctaccgtcatccgtcaccggcgattgtggcccgtaccgtgcgtattctacacacactgttgaccttggttaataagcaccgtaactgcgataaattcgaggtaaacacccagagcgtagcgtacctggccgccctattgacggtgagcgaagaagtccgtagccgctgtagcttgaagcaccgcaagtccctgctcctgacggacattagtatggagaacgtaccgatggacacgtatcccatccatcatggtgacccgagctaccgcacccttaaggaaacccaaccttggagtagcccgaagggctccgaaggttatctggcagcgacttacccgacagtgggtcaaacgtctcctcgtgcacggaaaagcatgagcctagacatgggccagccgagccaggcgaacaccaagaagctgctgggcacacgtaagtccttcgaccacttaatctctgataccaaagctccgaagcgtcaggagatggaaagcggcataaccacaccgccgaaaatgcgtcgtgttgccgagaccgactacgagatggaaacccaacgcatctcttcttcccagcagcacccgcatctgcgcaaggtcagcgttagcgaatccaacgtccttctggatgaagaagtgttgaccgatccgaaaattcaggcactgctgcttactgtcctcgcgaccctggtgaagtataccaccgacgaattcgatcagcgtatcctgtacgagtacctggcggaggcgagtgttgttttcccgaaagtgttcccggtagtgcacaacttgctggactccaagattaacaccctgttgagcctctgccaagatccgaatctgttaaacccgattcacgggatcgtacagagcgttgtctaccatgaagaatctccaccacagtatcagacgagttatctccagtcgttcggttttaacggcctttggcgctttgctggcccgttcagcaagcagactcaaattccagattatgcagaactgattgtcaagttcctggacgccctgattgacacgtatctgccgggtattgacgaggagaccagcgaggaaagcctgctgacccctaccagcccgtacccgccggcactgcagagccagctgagcataaccgcgaacttaaacctctctaacagcatgaccagcctggcgacctctcagcacagcccgggcattgacaaggagaacgtggagctctctccgaccaccggtcattgcaattccgggcgtacccgccacggctccgctagccaggtacaaaaacagcgttccgccggcagcttcaagcgcaacagcatcaaaaaaatcgtg

SEQ ID NO: 3 - DNA, 8517 bp, synthetic construct
  source 1..8517: mol_type = other DNA
atggcagctcacaggcccgtagaatgggttcaagcagttgtttctcgattcgacgaacaactgcctatcaaaaccggccagcaaaacacccataccaaagtctcgaccgagcataataaggagtgcctaatcaacattagtaagtacaaattcagcctggtcatctccggtctaaccacgatcctgaaaaacgtgaacaacatgcgtatctttggcgaagcggcggagaaaaacctgtacttgagccaactgattattctggacactttagagaagtgcctcgccggtcaaccaaaggacaccatgcgtttggacgaaacgatgctggtaaagcaattgctcccggagatctgccattttcttcacacctgccgtgagggtaatcagcacgcagcggagctacgtaactcggcgagcggtgtgctgttcagcttgagctgcaacaacttcaacgccgtcttctctcgtatcagcacgcgtcttcaggagctaaccgtgtgcagcgaggacaacgtggatgtgcatgatatcgagctcctgcagtacattaacgtggattgcgcaaagttgaagcgtttgctaaaagaaaccgcatttaagttcaaggcgctgaaaaaagtcgctcaactcgcggtgattaactcgctagaaaaggccttctggaattgggtggaaaactacccagatgaatttaccaagttataccagattccgcagactgatatggcggagtgcgcagaaaagcttttcgatctggtagatggctttgcggagagcaccaaacgtaaagcggccgtttggcctctgcaaattattttgttaattctatgcccggaaattatccaagatatctcaaaggacgttgtcgacgagaataacatgaataagaaattgttcctggactcactgcggaaggcgctggcgggtcatggtggtagccgccaactgactgagagcgcagcgattgcgtgtgttaagctgtgcaaagcgagtacctacattaactgggaagataacagcgtgatctttctgctggtccagtctatggttgtggacctgaagaatctgctgttcaatccgagcaagccgttctctcgtggttcccaaccggcagacgttgacctcatgattgactgcctggtgagctgcttccgtattagtccgcacaacaaccagcactttaaaatctgcctggcgcaaaattccccgagcaccttccattatgttctggttaatagcttgcacaggatcattaccaattctgccctggactggtggccgaagatagatgcagtatactgccatagcgtggaactacggaacatgttcggcgaaaccctacataaggcggtgcaaggctgtggtgcgcacccggcgatccgtatggctccgtctcttaccttcaaagaaaaagtgacctctctcaagttcaaggaaaagccgactgacctggagacgcgtagctacaagtacctcctgctgagcatggtcaagttgattcacgccgatccgaagctgctgctgtgcaatccgcgcaaacagggtccggaaacccagggttctaccgctgaattgatcaccggcctggttcagttggtgccgcagagccatatgccggagatagcgcaggaggctatggaagcgctgttagttctgcatcagctggacagcattgacctgtggaaccccgatgctccggtggaaaccttctgggaaatctctagtcaaatgctattctacatctgcaagaagctgacttcacaccagatgctctcttccactgaaatacttaagtggctgcgcgaaatcctgatttgccgtaacaagtttctgctgaaaaataagcaggccgaccgtagcagctgtcattttctactgttctacggggtgggctgcgatataccttcaagcggcaatacgagccagatgagcatggaccacgaggaattgctgcgtaccccgggcgcgagtctccgcaaaggtaaaggcaactcctctatggatagcgcggcgggctgcagcggtaccccgccgatctgccgtcaggcacaaaccaagctggaagtcgctctgtacatgttcttatggaatccggatacggaagccgtgcttgtcgctatgtcgtgtttccgtcatctgtgtgaagaagcagacatccgctgcggtgtggatgaagtttcggttcacaatctgctgccgaattacaacacgtttatggagttcgcctccgttagcaatatgatgtctaccggcagagcggcgttgcaaaaacgcgttatggcactcctgcgtcgtattgagcaccctacggcaggcaacaccgaagcttgggaagacacccatgcaaaatgggaacaggcaaccaagctcatcctcaattatccgaaggccaagatggaagacggccaagccgcggagtcactgcacaagacaatcgttaagcgccgcatgagccacgtatccggcggtggctctatcgacctttccgacaccgactcattacaagaatggatcaacatgaccggattcttgtgcgcgttgggcggcgtgtgtctgcaacaacgcagcaactctggtttggccacctacagcccgcccatgggtccggtctctgaacgcaagggtagcatgatcagcgttatgagtagtgagggtaacgcggataccccggtatccaaatttatggatcgtttgctgagcctgatggtgtgcaaccacgagaaagtgggcctccagattagaacgaacgtaaaggacctggtcggtctggagttaagcccagcgctgtacccgatgctttttaataaacttaaaaataccatcagcaagttctttgactcccaaggtcaggttctgttgacggataccaacacccaatttgtggagcagacgatcgcgatcatgaagaacctgttggacaaccataccgaaggctcgagcgagcaccttggccaagcttcgattgagaccatgatgctgaacctggtccgttatgtgcgcgtgctgggtaacatggttcatgcgattcaaatcaaaacgaaactttgccagctggttgaggttatgatggctcgccgtgatgatctgtccttctgccaggagatgaaatttcgtaacaaaatggtggagtatctgacggactgggttatgggtacctccaatcaggcggcagatgatgacgttaagtgcctgacccgcgatctggatcaggcgagcatggaggcggtcgtttccctgctggctggcctacccctgcagccggaggagggcgacggcgtggagctgatggaggcgaaatcccagctgttcttgaaatactttaccctgttcatgaatctgctgaacgactgctcggaggtcgaggatgagtcggcgcaaactggcggtcgcaaacgtggtatgtctcgtcgtctggcgtcactcaggcactgcaccgttctagctatgagcaatcttctgaatgctaatgtggattcaggtctgatgcactccatcggcttgggttatcacaaagacctccaaacccgcgccaccttcatggaagtcttgacgaagatcctgcagcagggtaccgagtttgataccttagcggagacagtgttggcggaccgcttcgagcgcctggtcgagctggtgaccatgatgggcgatcagggtgaactaccgattgctatggctctggcgaacgtagttccgtgtagccagtgggatgaattagcgagggttctcgtgacactctttgactcgcgccacctgctgtaccagctgctctggaatatgtttagcaaggaggttgagttagccgacagcatgcagaccctcttccgcggtaatagcctggcgtccaagatcatgaccttttgcttcaaggtttacggtgcgacgtacctgcagaaactgctcgacccgctgctgcgcattgttatcacctcaagcgactggcagcacgtcagcttcgaagttgatccaaccagactggagccaagcgagtccctggaggaaaatcaacgcaacctgctgcagatgactgaaaaattcttccatgctatcatcagcagctccagcgagtttccgccacagttgcgttctgtgtgccattgcctttatcaggcgacctgccatagtctgctcaataaagcgacagtgaaagaaaagaaggagaacaagaaaagcgtagtaagtcaacgtttcccgcaaaatagcattggtgccgtggggagcgctatgtttctgcgttttattaatccagcaatcgtcagcccgtacgaagccggcatactggacaaaaagccgccgccgcgtattgaacgtggtctgaagctgatgagcaaaatcctgcagagcattgcgaatcacgtgctgtttacgaaggaagagcacatgagaccattcaatgattttgttaagagcaactttgacgcagcgcgtcggttcttcctggacattgcaagcgattgcccgacttctgatgcggttaaccatagcttatcgttcatttcggacggtaacgtgctggctttgcaccgtctgttatggaataatcaggaaaaaattggccaatatttatcctctaatcgtgatcacaaggctgttggccgtcgtccgtttgacaaaatggccacgctcctggcgtatctgggcccaccggagcataaaccggtggcggacacccattggtcaagcctgaacctgacctcaagtaagtttgaagaattcatgacccgccaccaggtgcacgaaaaagaagagttcaaggcgctcaaaactctgagcattttttatcaggctggaacctccaaagcaggcaacccgatcttttattacgtggcgcgtcgtttcaaaaccggtcagatcaatggtgatctgctcatctatcacgttttgttgaccctgaagccatactacgccaaaccgtacgaaattgtggtcgacttgacccataccggcccctctaatagattcaaaaccgactttctgtctaagtggtttgttgtgttcccgggtttcgcatatgataacgtgtctgcggtgtatatttacaactgtaactcctgggttcgtgaatataccaagtaccacgaaagactgctgacaggtctgaagggtagcaagcgtctggtttttattgattgcccgggtaaattggccgagcacatcgagcatgaacaacagaaattgccggcagcgaccctggctctggaggaggacctgaaggtcttccacaacgccctgaaattggcccataaggatacgaaggtgagcattaaggtagggtccacagcggtgcaagtgacctcggcagagcgcacgaaggttctgggtcagtccgttttcctgaacgatatctactatgctagcgaaattgaggaaatctgtctggtcgacgagaaccaatttactttgacgatcgctaatcagggtacccctctgaccttcatgcatcaggagtgcgaagcaattgttcagtcaatcatccacatccgtacccgttgggaactgagccaaccggatagcattccgcaacacaccaagattcgcccgaaagacgttccgggtactttacttaatatcgcgctgctgaacctgggttccagtgacccgtcactgcgttcggcagcgtataacctactctgcgcactgacctgcacctttaacctgaagatcgaaggtcagctgcttgagacttcaggcctctgcatcccggccaacaacaccttgttcatcgtgtcaatcagcaaaaccctggccgctaatgagccgcacttgaccctcgagttcctcgaggaatgcatttctggcttttccaaaagcagcatcgaactaaagcatttatgcttggagtacatgactccgtggctgagtaaccttgtgcgtttctgcaagcacaacgatgacgcgaaacgtcaacgcgtcaccgcgattctggataaactgattaccatgaccatcaatgaaaaacaaatgtatcccagcatccaggcgaaaatttggggttcactgggacaaatcaccgacctgctggacgttgtactggattcctttattaaaacgtcggctaccggtggtctcgggagcatcaaagcggaggttatggcagacactgcagttgctctggcgagcggtaacgtaaaattagtctccagcaaggttattggtcgtatgtgcaagattatagataaaacctgtctaagcccaacaccgactctggaacaacatctgatgtgggatgacattgcgatcctagcgcgttatatgctgatgctgagcttcaacaactcgctcgacgtagcggctcatctgccgtacctgtttcacgtggtaaccttcctggtcgcgactggtccgcttagtctgagggcaagcacccacggcttagtgattaacattatccacagtttatgtacctgcagccagctgcacttttcagaagaaaccaagcaagtgctgagactcagcctgaccgagttcagccttccgaaattctatctgctctttggcatttctaaggtaaagtccgctgcggttatcgcttttcgtagctcatatcgtgatcgttcgttcagcccgggcagctacgaacgtgaaacgtttgctcttaccagcttggagaccgtgaccgaggcgctgctcgaaattatggaagcctgtatgagagacatcccgacctgtaaatggctggaccagtggaccgagctcgcccaacgtttcgcctttcagtataacccgagcttacaaccccgcgctctggttgtattcggatgtatcagcaaacgtgttagccacggccagattaagcagatcattcgcattctgagcaaagcccttgaaagctgtttgaaaggtccggatacctacaattcccaggtgctaatcgaggctactgtgatagccctgaccaaactgcagccgctgctcaacaaagattctccgttacacaaagcattgttttgggttgcagtggcggtactgcagcttgatgaggttaacctctatagcgcgggcaccgcgctgctggaacagaatctacacaccctggacagccttagaatttttaacgataagagcccggaagaggtgttcatggcaatccgtaatccgcttgaatggcattgtaaacaaatggaccattttgttggcctgaactttaattctaacttcaactttgcgctggttggtcatttactcaaaggttaccgtcatccgtccccggcgatcgtcgctcgtaccgtccgtatcttgcacaccttgctcacccttgtgaataaacatcgtaattgtgataaatttgaggtgaatacgcagtccgttgcttacttggctgccctcctgacagtttcggaagaggttcgttctcgttgttctctcaagcaccgcaaatccctgttgttaactgatatctctatggaaaacgtgccgatggatacctacccgattcatcatggcgacccgtcctaccgtaccttgaaagagacccagccttggagctcaccgaagggttctgagggctatctggcggctacatacccgaccgtggggcaaaccagtccgcgcgcccgtaagtctatgtctttggacatgggtcaaccatcccaagcgaacaccaagaagctgctgggcacacgtaaatccttcgaccacttgatcagcgataccaaagctccgaagcgccaagaaatggaaagcggtatcacaaccccgccgaagatgcgtcgtgttgcggaaaccgactatgaaatggaaacccagcgtatctctagcagtcagcagcacccgcacttgagaaaagtgagcgtttctgaatccaacgtactcctggacgaagaggttctgactgacccgaaaattcaagctctattactgacggtactggcaaccctggtgaaatataccaccgatgagtttgatcaacgcattctgtacgaatacctcgcggaggcaagcgtggtgtttccgaaagttttcccggttgttcacaacttgttggattccaagattaacactctactgagtttatgtcaggatccgaacctcctgaacccgatccacggcatcgtgcagagcgtcgtgtatcatgaggaaagtcctccgcagtatcagacttcttacctgcaaagctttggttttaacggtttgtggcgttttgctggtccgttcagcaaacaaacccagatcccggactacgcagagctaatcgttaaattcctggacgcacttattgacacttatttgccgggtatcgacgaagaaacgagcgaagagtcgctgctgactccaacctcgccgtatccgccggcactacaatcccaactgtcaatcacggcgaatctcaacctgtccaacagcatgaccagtcttgcgacgagtcagcacagccccggcattgacaaggaaaatgttgagttatctccaaccaccggccactgcaattctggtcgtacccggcacggctctgcgtcgcaggttcaaaaacagcgttccgcgggctcctttaagcgaaactccattaaaaaaatcgtc

SEQ ID NO: 4 - protein, 2839 aa, synthetic construct
MAAHRPVEWVQAVVSRFDEQLPIKTGQQNTHTKVSTEHNKECLINISKYKFSLVISGLTTILKNVNNMRIFGEAAEKNLYLSQLIILDTLEKCLAGQPKDTMRLDETMLVKQLLPEICHFLHTCREGNQHAAELRNSASGVLFSLSCNNFNAVFSRISTRLQELTVCSEDNVDVHDIELLQYINVDCAKLKRLLKETAFKFKALKKVAQLAVINSLEKAFWNWVENYPDEFTKLYQIPQTDMAECAEKLFDLVDGFAESTKRKAAVWPLQIILLILCPEIIQDISKDVVDENNMNKKLFLDSLRKALAGHGGSRQLTESAAIACVKLCKASTYINWEDNSVIFLLVQSMVVDLKNLLFNPSKPFSRGSQPADVDLMIDCLVSCFRISPHNNQHFKICLAQNSPSTFHYVLVNSLHRIITNSALDWWPKIDAVYCHSVELRNMFGETLHKAVQGCGAHPAIRMAPSLTFKEKVTSLKFKEKPTDLETRSYKYLLLSMVKLIHADPKLLLCNPRKQGPETQGSTAELITGLVQLVPQSHMPEIAQEAMEALLVLHQLDSIDLWNPDAPVETFWEISSQMLFYICKKLTSHQMLSSTEILKWLREILICRNKFLLKNKQADRSSCHFLLFYGVGCDIPSSGNTSQMSMDHEELLRTPGASLRKGKGNSSMDSAAGCSGTPPICRQAQTKLEVALYMFLWNPDTEAVLVAMSCFRHLCEEADIRCGVDEVSVHNLLPNYNTFMEFASVSNMMSTGRAALQKRVMALLRRIEHPTAGNTEAWEDTHAKWEQATKLILNYPKAKMEDGQAAESLHKTIVKRRMSHVSGGGSIDLSDTDSLQEWINMTGFLCALGGVCLQQRSNSGLATYSPPMGPVSERKGSMISVMSSEGNADTPVSKFMDRLLSLMVCNHEKVGLQIRTNVKDLVGLELSPALYPMLFNKLKNTISKFFDSQGQVLLTDTNTQFVEQTIAIMKNLLDNHTEGSSEHLGQASIETMMLNLVRYVRVLGNMVHAIQIKTKLCQLVEVMMARRDDLSFCQEMKFRNKMVEYLTDWVMGTSNQAADDDVKCLTRDLDQASMEAVVSLLAGLPLQPEEGDGVELMEAKSQLFLKYFTLFMNLLNDCSEVEDESAQTGGRKRGMSRRLASLRHCTVLAMSNLLNANVDSGLMHSIGLGYHKDLQTRATFMEVLTKILQQGTEFDTLAETVLADRFERLVELVTMMGDQGELPIAMALANVVPCSQWDELARVLVTLFDSRHLLYQLLWNMFSKEVELADSMQTLFRGNSLASKIMTFCFKVYGATYLQKLLDPLLRIVITSSDWQHVSFEVDPTRLEPSESLEENQRNLLQMTEKFFHAIISSSSEFPPQLRSVCHCLYQATCHSLLNKATVKEKKENKKSVVSQRFPQNSIGAVGSAMFLRFINPAIVSPYEAGILDKKPPPRIERGLKLMSKILQSIANHVLFTKEEHMRPFNDFVKSNFDAARRFFLDIASDCPTSDAVNHSLSFISDGNVLALHRLLWNNQEKIGQYLSSNRDHKAVGRRPFDKMATLLAYLGPPEHKPVADTHWSSLNLTSSKFEEFMTRHQVHEKEEFKALKTLSIFYQAGTSKAGNPIFYYVARRFKTGQINGDLLIYHVLLTLKPYYAKPYEIVVDLTHTGPSNRFKTDFLSKWFVVFPGFAYDNVSAVYIYNCNSWVREYTKYHERLLTGLKGSKRLVFIDCPGKLAEHIEHEQQKLPAATLALEEDLKVFHNALKLAHKDTKVSIKVGSTAVQVTSAERTKVLGQSVFLNDIYYASEIEEICLVDENQFTLTIANQGTPLTFMHQECEAIVQSIIHIRTRWELSQPDSIPQHTKIRPKDVPGTLLNIALLNLGSSDPSLRSAAYNLLCALTCTFNLKIEGQLLETSGLCIPANNTLFIVSISKTLAANEPHLTLEFLEECISGFSKSSIELKHLCLEYMTPWLSNLVRFCKHNDDAKRQRVTAILDKLITMTINEKQMYPSIQAKIWGSLGQITDLLDVVLDSFIKTSATGGLGSIKAEVMADTAVALASGNVKLVSSKVIGRMCKIIDKTCLSPTPTLEQHLMWDDIAILARYMLMLSFNNSLDVAAHLPYLFHVVTFLVATGPLSLRASTHGLVINIIHSLCTCSQLHFSEETKQVLRLSLTEFSLPKFYLLFGISKVKSAAVIAFRSSYRDRSFSPGSYERETFALTSLETVTEALLEIMEACMRDIPTCKWLDQWTELAQRFAFQYNPSLQPRALVVFGCISKRVSHGQIKQIIRILSKALESCLKGPDTYNSQVLIEATVIALTKLQPLLNKDSPLHKALFWVAVAVLQLDEVNLYSAGTALLEQNLHTLDSLRIFNDKSPEEVFMAIRNPLEWHCKQMDHFVGLNFNSNFNFALVGHLLKGYRHPSPAIVARTVRILHTLLTLVNKHRNCDKFEVNTQSVAYLAALLTVSEEVRSRCSLKHRKSLLLTDISMENVPMDTYPIHHGDPSYRTLKETQPWSSPKGSEGYLAATYPTVGQTSPRARKSMSLDMGQPSQANTKKLLGTRKSFDHLISDTKAPKRQEMESGITTPPKMRRVAETDYEMETQRISSSQQHPHLRKVSVSESNVLLDEEVLTDPKIQALLLTVLATLVKYTTDEFDQRILYEYLAEASVVFPKVFPVVHNLLDSKINTLLSLCQDPNLLNPIHGIVQSVVYHEESPPQYQTSYLQSFGFNGLWRFAGPFSKQTQIPDYAELIVKFLDALIDTYLPGIDEETSEESLLTPTSPYPPALQSQLSITANLNLSNSMTSLATSQHSPGIDKENVELSPTTGHCNSGRTRHGSASQVQKQRSAGSFKRNSIKKIV

SEQ ID NO: 5 - protein, 2839 aa, synthetic construct; identical to SEQ ID NO: 4

SEQ ID NO: 6 - protein, 2836 aa, Homo sapiens
MAAHRPVEWVQAVVSRFDEQLPIKTGQQNTHTKVSTEHNKECLINISKYKFSLVISGLTTILKNVNNMRIFGEAAEKNLYLSQLIILDTLEKCLAGQPKDTMRLDETMLVKQLLPEICHFLHTCREGNQHAAELRNSASGVLFSLSCNNFNAVFSRISTRLQELTVCSEDNVDVHDIELLQYINVDCAKLKRLLKETAFKFKALKKVAQLAVINSLEKAFWNWVENYPDEFTKLYQIPQTDMAECAEKLFDLVDGFAESTKRKAAVWPLQIILLILCPEIIQDISKDVVDENNMNKKLFLDSLRKALAGHGGSRQLTESAAIACVKLCKASTYINWEDNSVIFLLVQSMVVDLKNLLFNPSKPFSRGSQPADVDLMIDCLVSCFRISPHNNQHFKICLAQNSPSTFHYVLVNSLHRIITNSALDWWPKIDAVYCHSVELRNMFGETLHKAVQGCGAHPAIRMAPSLTFKEKVTSLKFKEKPTDLETRSYKYLLLSMVKLIHADPKLLLCNPRKQGPETQGSTAELITGLVQLVPQSHMPEIAQEAMEALLVLHQLDSIDLWNPDAPVETFWEISSQMLFYICKKLTSHQMLSSTEILKWLREILICRNKFLLKNKQADRSSCHFLLFYGVGCDIPSSGNTSQMSMDHEELLRTPGASLRKGKGNSSMDSAAGCSGTPPICRQAQTKLEVALYMFLWNPDTEAVLVAMSCFRHLCEEADIRCGVDEVSVHNLLPNYNTFMEFASVSNMMSTGRAALQKRVMALLRRIEHPTAGNTEAWEDTHAKWEQATKLILNYPKAKMEDGQAAESLHKTIVKRRMSHVSGGGSIDLSDTDSLQEWINMTGFLCALGGVCLQQRSNSGLATYSPPMGPVSERKGSMISVMSSEGNADTPVSKFMDRLLSLMVCNHEKVGLQIRTNVKDLVGLELSPALYPMLFNKLKNTISKFFDSQGQVLLTDTNTQFVEQTIAIMKNLLDNHTEGSSEHLGQASIETMMLNLVRYVRVLGNMVHAIQIKTKLCQLVEVMMARRDDLSFCQEMKFRNKMVEYLTDWVMGTSNQAADDDVKCLTRDLDQASMEAVVSLLAGLPLQPEEGDGVELMEAKSQLFLKYFTLFMNLLNDCSEVEDESAQTGGRKRGMSRRLASLRHCTVLAMSNLLNANVDSGLMHSIGLGYHKDLQTRATFMEVLTKILQQGTEFDTLAETVLADRFERLVELVTMMGDQGELPIAMALANVVPCSQWDELARVLVTLFDSRHLLYQLLWNMFSKEVELADSMQTLFRGNSLASKIMTFCFKVYGATYLQKLLDPLLRIVITSSDWQHVSFEVDPTRLEPSESLEENQRNLLQMTEKFFHAIISSSSEFPPQLRSVCHCLYQATCHSLLNKATVKEKKENKKSVVSQRFPQNSIGAVGSAMFLRFINPAIVSPYEAGILDKKPPPRIERGLKLMSKILQSIANHVLFTKEEHMRPFNDFVKSNFDAARRFFLDIASDCPTSDAVNHSLSFISDGNVLALHRLLWNNQEKIGQYLSSNRDHKAVGRRPFDKMATLLAYLGPPEHKPVADTHWSSLNLTSSKFEEFMTRHQVHEKEEFKALKTLSIFYQAGTSKAGNPIFYYVARRFKTGQINGDLLIYHVLLTLKPYYAKPYEIVVDLTHTGPSNRFKTDFLSKWFVVFPGFAYDNVSAVYIYNCNSWVREYTKYHERLLTGLKGSKRLVFIDCPGKLAEHIEHEQQKLPAATLALEEDLKVFHNALKLAHKDTKVSIKVGSTAVQVTSAERTKVLGQSVFLNDIYYASEIEEICLVDENQFTLTIANQGTPLTFMHQECEAIVQSIIHIRTRWELSQPDSIPQHTKIRPKDVPGTLLNIALLNLGSSDPSLRSAAYNLLCALTCTFNLKIEGQLLETSGLCIPANNTLFIVSISKTLAANEPHLTLEFLEECISGFSKSSIELKHLCLEYMTPWLSNLVRFCKHNDDAKRQRVTAILDKLITMTINEKQMYPSIQAKIWGSLGQITDLLDVVLDSFIKTSATGGLGSIKAEVMADTAVALASGNVKLVSSKVIGRMCKIIDKTCLSPTPTLEQHLMWDDIAILARYMLMLSFNNSLDVAAHLPYLFHVVTFLVATGPLSLRASTHGLVINIIHSLCTCSQLHFSEETKQVLRLSLTEFSLPKFYLLFGISKVKSAAVIAFRSSYRDRSFSPGSYERETFALTSLETVTEALLEIMEACMRDIPTCKWLDQWTELAQRFAFQYNPSLQPRALVVFGCISKRVSHGQIKQIIRILSKALESCLKGPDTYNSQVLIEATVIALTKLQPLLNKDSPLHKALFWVAVAVLQLDEVNLYSAGTALLEQNLHTLDSLRIFNDKSPEEVFMAIRNPLEWHCKQMDHFVGLNFNSNFNFALVGHLLKGYRHPSPAIVARTVRILHTLLTLVNKHRNCDKFEVNTQSVAYLAALLTVSEEVRSRCSLKHRKSLLLTDISMENVPMDTYPIHHGDPSYRTLKETQPWSSPKGSEGYLAATYPTVGQTSPRARKSMSLDMGQPSQANTKKLLGTRKSFDHLISDTKAPKRQEMESGITTPPKMRRVAETDYEMETQRISSSQQHPHLRKVSVSESNVLLDEEVLTDPKIQALLLTVLATLVKYTTDEFDQRILYEYLAEASVVFPKVFPVVHNLLDSKINTLLSLCQDPNLLNPIHGIVQSVVYHEESPPQYQTSYLQSFGFNGLWRFAGRFQSKHKFQTMLSLLLSFLMPLTRTCLELMKKPVKNPSLPHLLTLLHCRASLVSLPTLTFLIPPHLQLPSIPQESTRRTLNSPLPLATVTVDELATDPQAKCRSKEALAVSNVIALRRSC

(19) United States
(12) Patent Application Publication
(10) Pub. No.: US 2026/0297143 A1
(43) Pub. Date: Oct. 1, 2026

(54) MESSENGER RNA ENGINEERED THERAPEUTICS FOR TREATING GENETIC DISORDERS (MeET)

(71) Applicant: Board of Regents, the University of Texas System, Austin, TX (US)

(72) Inventors: Mohammed Maniruzzaman, Austin, TX (US); Leela Raghava Jaidev Chakka, Austin, TX (US); Niloofar Heshmati Aghda, Austin, TX (US)

(21) Appl. No.: 19/147,918

(22) PCT Filed: Jan. 31, 2024
(86) PCT No.: PCT/US2024/013699

§ 371 Date: Jul. 14, 2025

Related U.S. Application Data
(60) Provisional application No. 63/442,668, filed on Feb. 1, 2023.

(51) Int. Cl. C07K 14/47; A61K 9/51; A61K 38/17; A61K 48/00; A61P 35/00

(52) U.S. Cl. CPC C07K 14/4703; A61K 9/5123; A61K 38/1709; A61K 48/0041; A61K 48/005; A61P 35/00

(57) ABSTRACT

Described herein are compositions and methods for treatment of conditions relating to expression and production of neurofibromin proteins. The disclosed compositions include nucleic acid molecules (e.g., messenger RNA, mRNA) providing optimized sequences, such as encoding for neurofibromin proteins or related derivatives or fragments thereof and useful for treating conditions associated with neurofibromatosis type 1. The disclosed compositions may be implemented in the form of nanoparticles including the nucleic acid molecules and suspended in a suitable carrier, such as an aqueous emulsion of a cationic lipid complex. The particle emulsion may be generated using an extrusion-based printing process. Administration of the disclosed compositions can result in expression of neurofibromin proteins or related derivatives or fragments thereof in mammals.

### CROSS REFERENCE TO RELATED APPLICATIONS

This application claims the benefit of and priority to U.S. Provisional Application No. 63/442,668, filed on Feb. 1, 2023, which is hereby incorporated by reference in its entirety.

### REFERENCE TO BIOLOGICAL SEQUENCE DISCLOSURE

The instant application contains a Sequence Listing which has been submitted electronically in XML file format and is hereby incorporated by reference in its entirety. Said XML copy, created on Jan. 22, 2024, is named 093331-1418967_8050 MAN_ST26.xml and is 46,969 bytes in size.

### FIELD

This invention is in the field of biotechnology and mRNA therapeutics. This invention relates generally to the application of a designed and engineered mRNA molecule of a targeted gene and its encapsulation into lipid particles using an extrusion-based printing process integrated with engineered print heads and precipitation, emulsion, and gelation based methods.

### BACKGROUND

Genetic disorders are very difficult to treat using existing treatment modalities. The drugs developed for rare genetic disorders are referred to as orphan drugs. The traditional manufacturing methods employed by the pharmaceutical industry are not cost-effective to produce these drugs in limited quantities, rendering high costs. This, in turn, imposes a huge burden on the healthcare industry.

According to the National Organization of Rare Disorders, Neurofibromatosis type 1 is a rare genetic disorder affecting 1 in every 3000 Americans every year. Treatment for the disorder depends on the symptoms for the type of Neurofibromatosis (NF) that the patient experiences. Currently, there is no complete cure for Neurofibromatosis type 1 (NF1). Several disease targets for the treatment have been under investigation in various clinical trials. Koselugo (Selumetibib), manufactured by AstraZeneca in 2020, is the first FDA-approved drug to treat the disease in patients that are 2 years of age or older. The drug, Koselugo (Selumetibib), is a kinase inhibitor blocking an enzyme responsible for tumor growth. This drug causes serious side effects such as loss of vision, diarrhea, skin rash, and muscle problems to list a few. Though several drugs are under study, none of them have reached the stage of commercialization, there is no effective therapy for NF1 to date.

According to the neurofibromatosis clinics association, around 100 million Americans will benefit from the NF1 research. Hence there is an urgent need to research and develop novel new molecules with innovative and state-of-the-art technologies for NF1.

### SUMMARY

The pharmacology of mRNA offers the development of cellular immune responses, which are very useful to treat diseases such as cancer. The synthesis of clinical-grade mRNA (FIG. 1, Panel A) uses an optimized In Vitro Transcription (IVT) synthesis process (FIG. 1, Panel B). The process utilizes a DNA template and various enzymes in a cell-free system ultimately producing a cost-effective and therapeutically viable mRNA. The IVT process facilitates control over every element of mRNA structure which can be optimized for better protein expression. The synthesized and optimized mRNA can be complexed with lipid moieties or commercial transfection agents for use in employment or evaluating their anticancer activity. Upon administration (FIG. 1, Panel C), the mRNA complexes can be taken up by the endosomes of the cell.

The lipid moieties prevent the degradation of mRNA in endosomes and help its escape into the cytoplasm. The escaped mRNA can be translated to protein in the cell cytoplasm. The primary protein undergoes secondary post-translational modifications and can be presented as a complete protein to the surrounding cells. The synthesized neurofibromin protein can regulate cell growth through the Ras signaling pathway. The cells eventually undergo apoptosis and then cell death.

Described herein is the novel application of engineered mRNA of genes responsible for rare genetic disorders, such as those related to Neurofibromatosis type 1 (NF1), wherein these engineered mRNA are functionally viable and able to translate into proteins. The engineered mRNA described herein may be transcribed into multiple copies using in vitro transcription methods. The purified mRNA can then be mixed with an optimized Nitrogen/Phosphorous (N/P) ratio of 8-10 of lipid composition, wherein a mixture of the purified mRNA and lipid composition may be extruded using SMART extrusion (as described in PCT/US2022/036336, hereby incorporated by reference) through the MAGIC nozzle (as described in U.S. provisional application 63/392,795, hereby incorporated by reference) to produce mRNA loaded lipid nanoparticles. These nanoparticles may be stored at regular storage conditions of 4 degrees Celsius (4° C.), for example. The nanoparticles may optionally include stable cryogenic agents such as sucralose.

The methods of producing the nanoparticles described herein may produce the nanoparticles continuously based on the need or on-demand. The facile and improved synthesis mechanism described herein offers cost effective treatment strategies to the otherwise impossible-to-treat rare genetic disorders.

Without wishing to be bound by any particular theory, there can be discussion herein of beliefs or understandings of underlying principles relating to the invention. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1 provides an image showing the elements of mRNA (Panel A) and the in vitro transcription (IVT) synthesis of mRNA from a double strand DNA construct with subsequent steps of RNA transcription (ssRNA), 5′ cap addition and poly A tail addition (mature RNA) (Panel B) and Mechanism of mRNA uptake, translation, and activity in NF1 cancer cells (Panel C).

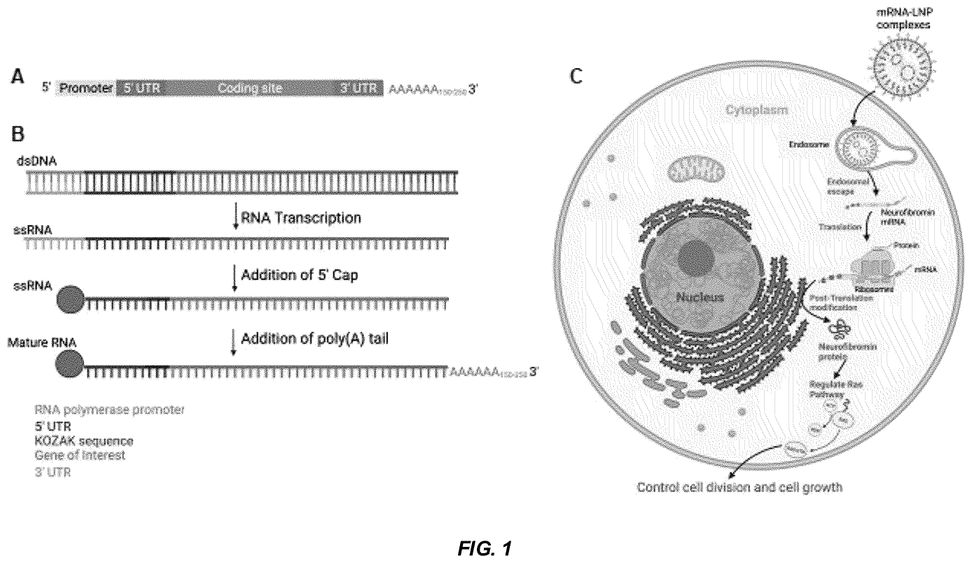

FIG. 2 provides Particle size analysis (Panel A) and Poly Dispersity Index (PDI) (Panel B) of the lipid mRNA particles prepared at different N/P ratios.

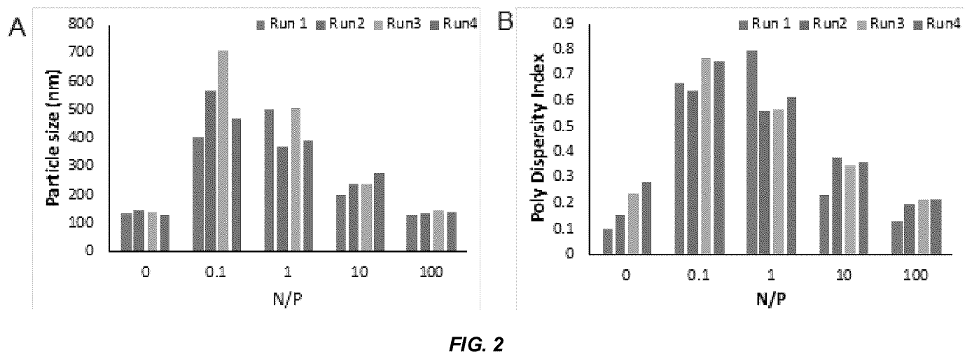

FIG. 3 provides a gel retardation assay performed to identify the optimal LNP-mRNA complexation at appropriate N/P ratios. The agarose gel image under UV transilluminator shows the migration of free RNA as a fluorescence band across the gel from the well whereas the complexed RNA with lipid components stay in the well, showing bright fluorescence.

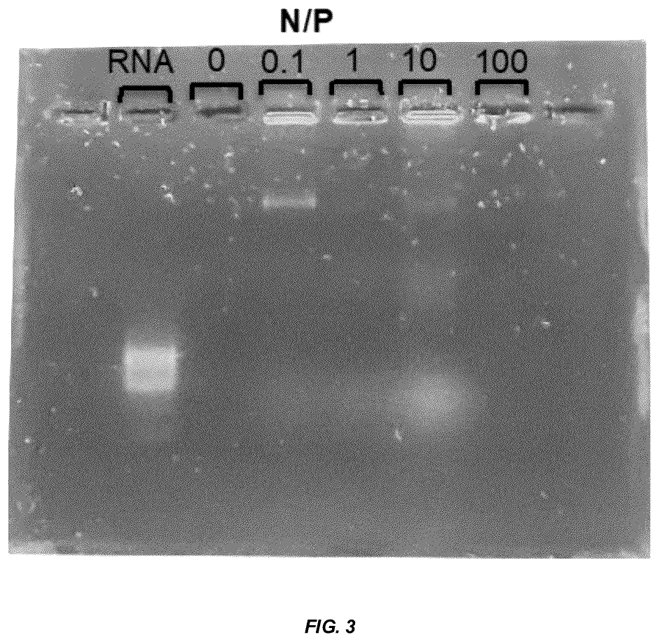

FIG. 4 provides a luminescence assay to determine the luciferase quantities after treating MG63 cells with luciferase RNA loaded lipid nanoparticles and incubated for 24 hours. The data represented are mean values.

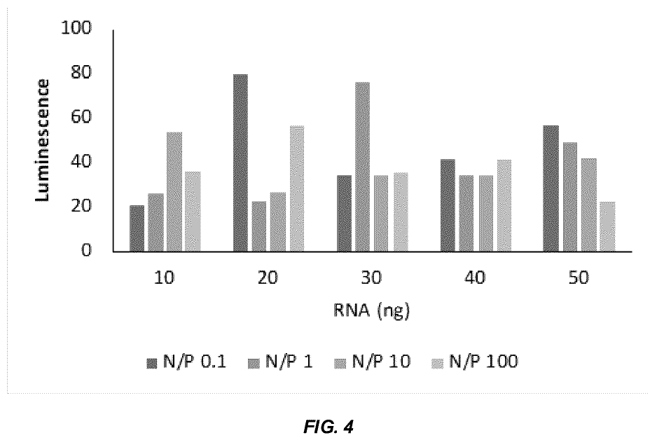

FIG. 5 provides a graph showing the treatment of osteosarcoma cells (MG63) with the mRNA-Lipid particles to see the toxicity activity. The results are the MTT assay (using the 3-(4,5-dimethylthiazol-2-yl)-2,5-diphenyl-2H-tetrazolium bromide reagent) showing the cell proliferation. The data are mean±standard deviation.

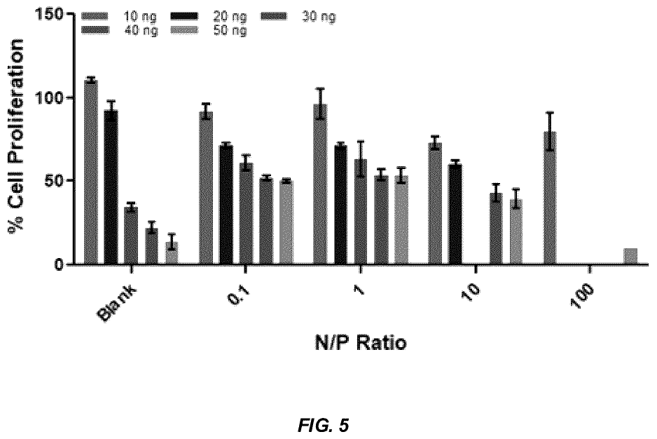

FIG. 6 shows cell viability of MG63 osteosarcoma cells after incubation of LNP-mRNA (Erythropoietin (EPO)) of different N/P ratio

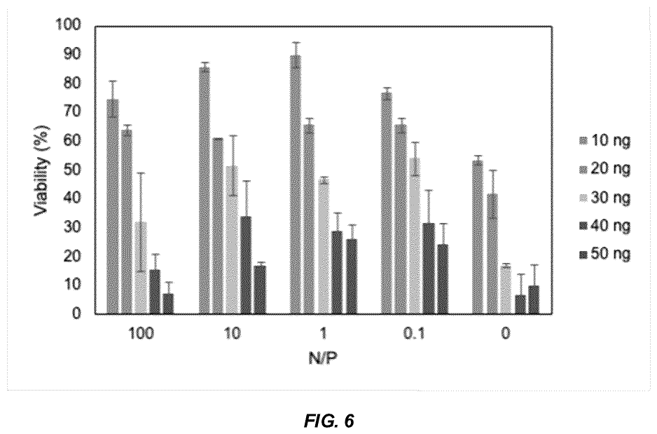

FIG. 7 shows particle size analysis of LNP-mRNA (Erythropoietin (EPO)) with different N/P ratio prepared using SMART.

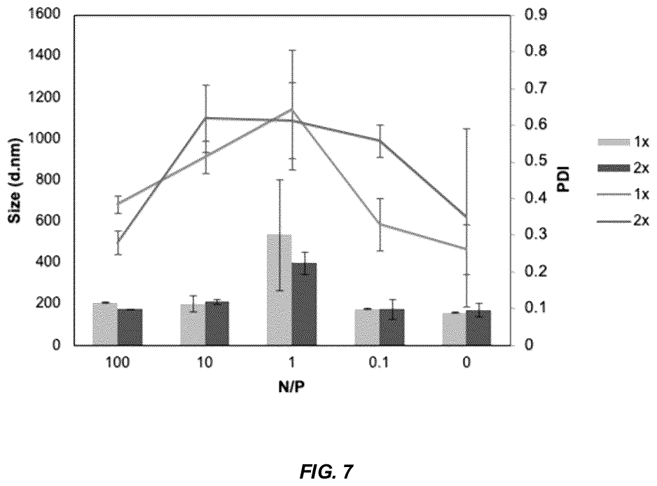

### DETAILED DESCRIPTION

### I. Overview and Definitions

In general the terms and phrases used herein have their art-recognized meaning, which can be found by reference to standard texts, journal references and contexts known to those skilled in the art. The following definitions are provided to clarify their specific use in the context of the invention.

As used herein, the term “nucleotide,” in addition to referring to the naturally occurring ribonucleotide or deoxyribonucleotide monomers, shall herein be understood to refer to related structural variants thereof, including derivatives and analogs, that are functionally equivalent with respect to the particular context in which the nucleotide is being used (e.g., hybridization to a complementary base), unless the context clearly indicates otherwise.

As used herein, the term “amino acid”, refers to any monomeric unit that can be incorporated into a peptide, polypeptide, or protein. As used herein, the term “amino acid” includes the following twenty natural or genetically encoded alpha-amino acids: alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (H is or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). In cases where “X” residues are undefined, these should be defined as “any amino acid.” The structures of these twenty natural amino acids are shown in, e.g., Stryer et al., Biochemistry, 5th ed., Freeman and Company (2002), which is incorporated by reference. Additional amino acids, such as selenocysteine and pyrrolysine, can also be genetically coded for (Stadtman (1996) “Selenocysteine,” Annu Rev Biochem. 65:83-100 and Ibba et al. (2002) “Genetic code: introducing pyrrolysine,” Curr Biol. 12 (13): R464-R466, which are both incorporated by reference). The term “amino acid” also includes unnatural amino acids, modified amino acids (e.g., having modified side chains and/or backbones), and amino acid analogs.

As used herein, the term “polypeptide,” “peptide,” and “protein”, are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-natural amino acid polymers. As used herein, the terms encompass amino acid chains of any length, including full-length proteins, wherein the amino acid residues are linked by covalent peptide bonds.

As used herein, the term “nucleic acid”, “nucleic acid molecule” or “polynucleotide”, refers to a polymer that can be corresponded to a ribose nucleic acid (RNA) or deoxyribose nucleic acid (DNA) polymer, or an analog thereof. This includes polymers of nucleotides such as RNA and DNA, as well as synthetic forms, modified (e.g., chemically or biochemically modified) forms thereof, and mixed polymers (e.g., including both RNA and DNA subunits). Exemplary modifications include methylation, substitution of one or more of the naturally occurring nucleotides with an analog, internucleotide modifications such as uncharged linkages (e.g., methyl phosphonates, phosphotriesters, phosphoamidates, carbamates, and the like), pendent moieties (e.g., polypeptides), intercalators (e.g., acridine, psoralen, and the like), chelators, alkylators, and modified linkages (e.g., alpha anomeric nucleic acids and the like). Also included are synthetic molecules that mimic polynucleotides in their ability to bind to a designated sequence via hydrogen bonding and other chemical interactions. Typically, the nucleotide monomers are linked via phosphodiester bonds, although synthetic forms of nucleic acids can comprise other linkages (e.g., peptide nucleic acids as described in Nielsen et al. (Science 254:1497-1500, 1991). A nucleic acid molecule can be or can include, e.g., a chromosome or chromosomal segment, a vector (e.g., an expression vector), an expression cassette, a naked DNA or RNA polymer, the product of a polymerase chain reaction (PCR), an oligonucleotide, a probe, and a primer. A nucleic acid molecule can be, e.g., single-stranded, double-stranded, or triple-stranded and is not limited to any particular length. Unless otherwise indicated, a particular nucleic acid molecule can have a nucleotide sequence that comprises or encodes complementary sequences, in addition to any sequence explicitly indicated.

As used herein, a nucleic acid molecule is “operably linked”, when it is placed into a functional relationship with another nucleotide sequence. For example, a promoter or enhancer is operably linked to a coding sequence if it affects the transcription of the sequence; or a ribosome binding site is operably linked to a coding sequence if it is positioned so as to facilitate translation.

As used herein, the terms “identical” or percent “identity,” in the context of describing two or more polynucleotide or amino acid sequences, refer to two or more sequences or subsequences that are the same or have a specified percentage of amino acid residues or nucleotides that are the same. For example, a sequence can have at least 80% identity, preferably 85%, 90%, 91%, 92%, 93%, 94%, 95%, 96%, 97%, 98%, 99%, or 100% identity, to a reference sequence when compared and aligned for maximum correspondence over a comparison window, or designated region as measured using a sequence comparison algorithm or by manual alignment and visual inspection. Such sequences are then said to be “substantially identical.” With regard to polynucleotide sequences, this definition also refers to the complement of a test sequence. With regard to amino acid sequences, preferably, the identity exists over a region that is at least about 50 amino acids or nucleotides in length, or more preferably over a region that is 75-100 amino acids or nucleotides in length, or most preferably over the entirety of the query molecule.

As used herein, the term “percent sequence identity”, is determined by comparing two optimally aligned sequences over a comparison window, wherein the portion of the sequence in the comparison window can comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

As used herein, the term “similarity”, or “percent similarity,” in the context of two or more polypeptide sequences, refer to two or more sequences or subsequences that have a specified percentage of amino acid residues that are either the same or similar as defined by a conservative amino acid substitutions (e.g., 60% similarity, optionally 65%, 70%, 75%, 80%, 85%, 90%, or 95% similar over a specified region), when compared and aligned for maximum correspondence over a comparison window, or designated region as measured using one of the following sequence comparison algorithms or by manual alignment and visual inspection. Sequences are “substantially similar” to each other if they are at least 20%, at least 25%, at least 30%, at least 35%, at least 40%, at least 45%, at least 50%, or at least 55% similar to each other. Optionally, this similarly exists over a region that is at least about 50 amino acids in length, or more preferably over a region that is 75-100 amino acids in length, or most preferably over the entirety of the query molecule.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are commonly used, or alternative parameters can be designated. The sequence comparison algorithm then calculates the percent sequence identities or similarities for the test sequences relative to the reference sequence, based on the program parameters.

As used herein, a “comparison window”, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, for example, by the local homology algorithm of Smith and Waterman (Adv. Appl. Math. 2:482, 1970), by the homology alignment algorithm of Needleman and Wunsch (J. Mol. Biol. 48:443, 1970), by the search for similarity method of Pearson and Lipman (Proc. Natl. Acad. Sci. USA 85:2444, 1988), by computerized implementations of these algorithms (e.g., GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Example algorithms suitable for determining percent sequence identity and sequence similarity are the BLAST and BLAST 2.0 algorithms, which are described in Altschul et al. (Nuc. Acids Res. 25:3389-402, 1977), and Altschul et al. (J. Mol. Biol. 215:403-10, 1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-87, 1993). One measure of similarity provided by the BLAST algorithm is the smallest sum probability (P(N)), which provides an indication of the probability by which a match between two nucleotide or amino acid sequences would occur by chance. For example, a nucleic acid molecule is considered similar to a reference sequence if the smallest sum probability in a comparison of the test nucleic acid molecule to the reference nucleic acid molecule or sequence is less than about 0.2, typically less than about 0.01, and more typically less than about 0.001. Various algorithms and other tools are publicly available, such as the Expasy bioinformatics resource portal (expasy.org) of the Swiss Institute of Bioinformatics (SIB) provided by the Swiss Institute of Bioinformatics (SIB), Séverine Duvaud, Chiara Gabella, Frédérique Lisacek, Heinz Stockinger, Vassilios Ioannidis, Christine Durinx; Expasy, the Swiss Bioinformatics Resource Portal, as designed by its users. Nucleic Acids Research, 2021. DOI: 10.1093/nar/gks225.

As used herein, the term “expression vector” is a nucleic acid construct, generated recombinantly or synthetically, with a series of specified nucleic acid elements that permit transcription of a particular nucleic acid in a host cell, and optionally integration or replication of the expression vector in a host cell. The expression vector can be part of a plasmid, virus, or nucleic acid fragment, of viral or non-viral origin. Typically, the expression vector includes an “expression cassette,” which comprises a nucleic acid to be transcribed operably linked to a promoter. The term expression vector also encompasses naked RNA or mRNA operably linked to a promoter. In some examples, the promoter can be a T7 promoter or a CMV promoter.

### II. Polynucleotides Encoding Neurofibromin Protein

In some embodiments, nucleic acid molecules described herein encode a polypeptide comprising an amino acid sequence SEQ ID NO: 4. In some embodiments, nucleic acid molecules described herein encode a polypeptide comprising an amino acid sequence SEQ ID NO: 5.

In some embodiments, nucleic acid molecules described herein comprise a modified SEQ ID NO: 1, wherein the modified SEQ ID NO: 1 comprises removal of stop codons or removal of restriction enzyme sites [GCGATG] relative to wildtype, and wherein the nucleic acid molecules encode a neurofibromin protein or a neurofibromin protein derivative.

The term “wildtype” as used herein when referring to a polynucleotide sequence that encodes a polypeptide is the polynucleotide sequence in its natural, non-mutated, unchanged form. When referring to the wildtype sequence for the nucleotide sequence encoding a neurofibromin protein, the wildtype sequence is SEQ ID NO: 1.

In some embodiments, the nucleic acid molecule has a sequence with at least 80% sequence identity with a sequence selected from the group consisting of SEQ ID NO: 2 and SEQ ID NO: 3. For example, the sequence has at least 80%, 85%, 90%, 95%, 99%, or 100% homology with SEQ ID NO: 2 or SEQ ID NO: 3. In some embodiments, the sequence is SEQ ID NO: 2. In some embodiments, the sequence is SEQ ID NO: 3. In some embodiments, the nucleic acid molecule is an mRNA or a fragment thereof. In some embodiments, the mRNA or fragment thereof has a sequence of at least 80% sequence identity with SEQ ID NO: 2 (e.g., at least 80%, 85%, 90%, 95%, 99%, or 100%). In some embodiments, the mRNA or fragment thereof has a sequence of at least 80% sequence identity with SEQ ID NO: 3 (e.g., at least 80%, 85%, 90%, 95%, 99%, or 100%).

### III. Compositions

In some embodiments, a composition as described herein comprises a nucleic acid molecule encoding a neurofibromin protein or fragment thereof that has at least 80% homology with amino acid sequence as depicted in SEQ ID NO: 4 or SEQ ID NO: 5, and a cationic lipid complex. In some embodiments, the nucleic acid molecule has at least 80% sequence identity with SEQ ID NO: 2, (e.g., the nucleic acid molecule may have at least 80%, 85%, 90%, 95%, 99%, or 100% sequence identity with SEQ ID NO: 2). In some embodiments, the nucleic acid molecule has 100% sequence identity with SEQ ID NO: 2. In some embodiments, the nucleic acid molecule has at least 80% sequence identity with SEQ ID NO: 3, (e.g., the nucleic acid molecule may have at least 80%, 85%, 90%, 95%, 99%, or 100% sequence identity with SEQ ID NO: 3). In some embodiments, the nucleic acid molecule has 100% sequence identity with SEQ ID NO: 3. In some embodiments, the nucleic acid molecule is an mRNA.

In certain non-limiting examples, the cationic lipid complex may include one or more cationic lipids. The term “cationic lipid” as used herein, refers to a lipid with a head group with a permanent positive charge. In some examples, the one or more cationic lipids may include 1,2-Dioleoyl-3-trimethylammonium propane (“DOTMA”), 2-dioleoyl-3-trimethylammonium-propane (“DOTAP”), 2,3-dioleyloxy-N-[2-(sperminecarboxamido)ethyl]-N,N-dimethyl-1-propanaminium (“DOSPA”), 1,2-dioleoyl-sn-glycero-3-ethylphosphocholine (“EPC”), N-methyl-4-(dioleyl)methylpyridinium (“SAINT-2”), 1,2-dioleoyl-sn-glycero-3-[phospho-1-serine] (“PS”), 3β-[N—(N′,N′-dimethylaminoethane)-carbamoyl]cholesterol (“DC-Chol”), Dimethyldioctadecylammonium bromide (“DDAB”), or a combination thereof. In some examples, pharmaceutically acceptable salts of the cationic lipids may be included in the cationic lipid complex.

In some embodiments, the composition further comprises one or more cationic polymers or a pharmaceutically acceptable salt thereof. The term “cationic polymer” as used herein refers to a polymer that bears a positive charge, which can be either present in the polymer backbone, in the polymer side chains, or a combination thereof. In some embodiments, the cationic polymer contains a primary, secondary, or tertiary amine functional group that can be protonated. In some embodiments, the cationic polymer may contain an ammonium ion. In some embodiments, the cationic polymer may include a phosphonium ion. The cationic polymer may include 1,2-dioleoyl-3-trimethylammonium-propane (“DOTAP”), 1,2-distearoyl-sn-glycero-3-phosphoethanolamine (“DPSE”), or a combination thereof. In some embodiments, one or more cationic polymers may be present. In some examples, the one or more cationic polymers is polyethyleneimine (PEI), functionalized polyethyleneimine, a poly-β-amino-ester (“PBAE”), functionalized poly-β-amino-ester, a branched poly-amino-ester (“PAE”), a polymethacrylate, a poly [2-(N,N-dimethylamino)ethyl methacrylate (“PDMAEMA”), a poly(L-lysine) (“PLL”), a cationic cyclodextrin-based polymer, a chitosan, a cationic dextran cationic cellulose, a gelatine, a dendrimer, a polysaccharide-based polymer, a polydisulfide amine, a polyamine, a polyamidoamine, or a combination thereof.

In some embodiments, the composition further comprises one or more ionic lipids or a pharmaceutically acceptable salt thereof. The term “ionic lipid” as used herein refers to lipids that are protonated at low pH and have a positive charge, but remain neutral at physiological pH. In some embodiments, the one or more ionic lipids may be 4-(dimethylamino)-butanoic acid, (10Z,13Z)-1-(9Z,12Z)-9,12-octadecadien-1-yl-10,13-nonadecadien-1-yl ester (“DLin-MC3-DMA”), ([(4-hydroxybutyl) azanediyl]di(hexane-6,1-diyl) bis(2-hexyldecanoate)) (“ALC-0315”), 8-[(2-hydroxyethyl) [6-oxo-6-(undecyloxy) hexyl]amino]-octanoic acid, 1-octylnonyl ester (“Lipid H (SM-102)”), ethyl 5,5-di((Z)-heptadec-8-en-1-yl)-1-(3-(pyrrolidin-1-yl) propyl)-2,5-dihydro-1H-imidazole-2-carboxylate (“A2-Iso5-2DC18”), bis(2-(dodecyldisulfanyl)ethyl) 3,3′-((3-methyl-9-oxo-10-oxa-13,14-dithia-3,6-diazahexacosyl) azanediyl)dipropionate (“BAME-016B”), decyl(2-(dioctylammonio)ethyl) phosphate (“9A1P9”), 1,1′-((2-(4-(2-((2-(bis(2-hydroxydodecyl)amino)ethyl) (2-hydroxydodecyl)amino)ethyl) piperazin-1-yl)ethyl) azanediyl)bis(dodecan-2-ol) (“C12-200”), 3,6-bis(4-(bis(2-hydroxydodecyl)amino)butyl) piperazine-2,5-dione (“cKK-E12”), (((3,6-dioxopiperazine-2,5-diyl)bis(butane-4, 1-diyl))bis(azanetriyl))tetrakis(ethane-2,1-diyl) (9Z,9′Z,9″Z,9″Z,12Z,12′Z,12″Z,12″Z)-tetrakis(octadeca-9,12-dienoate) (“OF-Deg-Lin”), tetrakis(8-methylnonyl) 3,3′,3″,3″-(((methylazanediyl)bis(propane-3,1 diyl))bis (azanetriyl))tetrapropionate (“306Oᵢ₁₀”), N¹,N³,N⁵-tris(3-(didodecylamino) propyl)benzene-1,3,5-tricarboxamide (“TT3”), hexa (octan-3-yl) 9,9′,9″,9′″,9″″,9′″″-((((benzene-1,3,5-tricarbonyl)yris(azanediyl)) tris (propane-3,1-diyl)) tris(azanetriyl)) hexanonanoate (“FTT5”), or a combination thereof.

In some embodiments, the composition further comprises one or more other lipids. The one or more other lipids may include cholesterol, DC-Cholesterol, BHEM-Cholesterol, distearoylphosphatidylcholine (“DSPC”), 1,2-dimyristoyl-rac-glycero-3-methoxypolyethylene glycol-2000 (“DMG-PEG₂₀₀₀”), 1,2-dioleoyl-sn-glycero-3-phosphoethanolamine (“DOPE”), α-[2-(ditetradecylamino)-2-oxoethyl]-ω-methoxy-poly (oxy-1,2-ethanediyl) (“ALC-0159”), β-sitosterol, 1,2-distearoyl-rac-glycero-3-methoxypolyethylene glycol-2000 (“DSG-PEG₂₀₀₀”), or a combination thereof.

Certain embodiments of the present disclosure relate to the therapeutic use of the nucleic acid molecules described herein. For therapeutic use, the nucleic acid molecules may be provided in the form of compositions which comprise the nucleic acid molecule and a pharmaceutically acceptable carrier or diluent. The compositions may be prepared by known procedures using well-known and readily available ingredients and may be formulated for administration to a subject by, for example, oral (including, for example, buccal or sublingual), topical, parenteral, rectal or vaginal routes, or by inhalation or spray. The term “parenteral” as used herein includes injection or infusion by subcutaneous, intradermal, intra-articular, intravenous, intramuscular, intravascular, intrasternal or intrathecal routes.

The composition may be formulated in a format suitable for administration to a subject by the chosen route, for example, as a syrup, elixir, tablet, troche, lozenge, hard or soft capsule, pill, suppository, oily or aqueous suspension, dispersible powder or granule, emulsion, injectable or solution. Compositions may be provided as unit dosage formulations.

Pharmaceutically acceptable carriers are generally non-toxic to recipients at the dosages and concentrations employed. Examples of such carriers include, but are not limited to, buffers such as phosphate, citrate, and other organic acids; antioxidants such as ascorbic acid and methionine; preservatives such as octadecyldimethylbenzyl ammonium chloride, hexamethonium chloride, benzalkonium chloride, benzethonium chloride, phenol, butyl alcohol, benzyl alcohol, alkyl parabens (such as methyl or propyl paraben), catechol, resorcinol, cyclohexanol, 3-pentanol and m-cresol; low molecular weight (less than about 10 amino acids) polypeptides; proteins such as serum albumin or gelatin; hydrophilic polymers such as polyvinylpyrrolidone; amino acids such as glycine, glutamine, asparagine, histidine, arginine or lysine; monosaccharides, disaccharides, and other carbohydrates such as glucose, mannose or dextrins; chelating agents such as EDTA; sugars such as sucrose, mannitol, trehalose or sorbitol; salt-forming counter-ions such as sodium; metal complexes such as Zn-protein complexes, and non-ionic surfactants such as polyethylene glycol (PEG).

In certain embodiments, the compositions may be in the form of a sterile injectable aqueous or oleaginous solution or suspension. Such suspensions may be formulated using suitable dispersing or wetting agents and/or suspending agents that are known in the art. The sterile injectable solution or suspension may comprise the nucleic acid molecule in a non-toxic parentally acceptable diluent or solvent. Acceptable diluents and solvents that may be employed include, for example, 1,3-butanediol, water, Ringer's solution or isotonic sodium chloride solution. In addition, sterile, fixed oils may be employed as a solvent or suspending medium. For this purpose, various bland fixed oils may be employed, including synthetic mono- or diglycerides. In addition, fatty acids such as oleic acid find use in the preparation of injectables. Adjuvants such as local anaesthetics, preservatives and/or buffering agents as known in the art may also be included in the injectable solution or suspension.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, PA (2000).

### Nanoparticle Compositions

In another aspect, a composition as described herein comprises nanoparticles. For example, the nanoparticle may comprise a nucleic acid molecule encoding a neurofibromin protein or fragment thereof that has at least 80% homology with amino acid sequence as depicted in SEQ ID NO: 4 or SEQ ID NO: 5, and a cationic lipid complex. In some embodiments, the nucleic acid molecule has at least 80% sequence identity with SEQ ID NO: 2, (e.g., the nucleic acid molecule may have at least 80%, 85%, 90%, 95%, 99%, or 100% sequence identity with SEQ ID NO: 2). In some embodiments, the nucleic acid molecule has 100% sequence identity with SEQ ID NO: 2. In some embodiments, the nucleic acid molecule has at least 80% sequence identity with SEQ ID NO: 3, (e.g., the nucleic acid molecule may have at least 80%, 85%, 90%, 95%, 99%, or 100% sequence identity with SEQ ID NO: 3). In some embodiments, the nucleic acid molecule has 100% sequence identity with SEQ ID NO: 3. In some embodiments, the nucleic acid molecule is an mRNA.

In certain non-limiting examples, the cationic lipid complex may include one or more cationic lipids or a pharmaceutically acceptable salt thereof. The term “cationic lipid” as used herein, refers to a lipid with a head group with a permanent positive charge. In some examples, the one or more cationic lipids may be 1,2-Dioleoyl-3-trimethylammonium propane (“DOTMA”), 2-dioleoyl-3-trimethylammonium-propane (“DOTAP”), 2,3-dioleyloxy-N-[2-(sperminecarboxamido)ethyl]-N,N-dimethyl-1-propanaminium (“DOSPA”), 1,2-dioleoyl-sn-glycero-3-ethylphosphocholine (“EPC”), N-methyl-4-(dioleyl)methylpyridinium (“SAINT-2”), 1,2-dioleoyl-sn-glycero-3-[phospho-1-serine] (“PS”), 3β-[N—(N′,N′-dimethylaminoethane)-carbamoyl]cholesterol (“DC-Chol”), Dimethyldioctadecylammonium bromide (“DDAB”), or a combination thereof.

In some embodiments, the nanoparticles further comprise one or more cationic polymers or a pharmaceutically acceptable salt thereof. The term “cationic polymer” as used herein refers to a polymer that bears a positive charge, which can be either present in the polymer backbone, in the polymer side chains, or a combination thereof. In some embodiments, the cationic polymer contains a primary, secondary, or tertiary amine functional group that can be protonated. In some embodiments, the cationic polymer may contain an ammonium ion. In some embodiments, the cationic polymer may include a phosphonium ion. The cationic polymer may include 1,2-dioleoyl-3-trimethylammonium-propane (“DOTAP”), 1,2-distearoyl-sn-glycero-3-phosphoethanolamine (“DPSE”), or a combination thereof. In some embodiments, one or more cationic polymers may be present. In some examples, the one or more cationic polymers is polyethyleneimine (PEI), functionalized polyethyleneimine, a poly-β-amino-ester (“PBAE”), functionalized poly-β-amino-ester, a branched poly-amino-ester (“PAE”), a polymethacrylate, a poly [2-(N,N-dimethylamino)ethyl methacrylate (“PDMAEMA”), a poly(L-lysine) (“PLL”), a cationic cyclodextrin-based polymer, a chitosan, a cationic dextran cationic cellulose, a gelatine, a dendrimer, a polysaccharide-based polymer, a polydisulfide amine, a polyamine, a polyamidoamine, or a combination thereof.

In some embodiments, the nanoparticles further comprise one or more ionic lipids or a pharmaceutically acceptable salt thereof. The term “ionic lipid” as used herein refers to lipids that are protonated at low pH and have a positive charge but remain neutral at physiological pH. In some embodiments, the one or more ionic lipids may include 4-(dimethylamino)-butanoic acid, (10Z,13Z)-1-(9Z,12Z)-9,12-octadecadien-1-yl-10,13-nonadecadien-1-yl ester (“DLin-MC3-DMA”), ([(4-hydroxybutyl) azanediyl]di(hexane-6,1-diyl)bis(2-hexyldecanoate)) (“ALC-0315”), 8-[(2-hydroxyethyl) [6-oxo-6-(undecyloxy) hexyl]amino]-octanoic acid, 1-octylnonyl ester (“Lipid H (SM-102)”), ethyl 5,5-di((Z)-heptadec-8-en-1-yl)-1-(3-(pyrrolidin-1-yl) propyl)-2,5-dihydro-1H-imidazole-2-carboxylate (“A2-Iso5-2DC18”), bis(2-(dodecyldisulfanyl)ethyl) 3,3′-((3-methyl-9-oxo-10-oxa-13,14-dithia-3,6-diazahexacosyl) azanediyl)dipropionate (“BAME-016B”), decyl(2-(dioctylammonio)ethyl) phosphate (“9A1P9”), 1,1′-((2-(4-(2-((2-(bis(2-hydroxydodecyl)amino)ethyl) (2-hydroxydodecyl)amino)ethyl) piperazin-1-yl)ethyl) azanediyl)bis(dodecan-2-ol) (“C12-200”), 3,6-bis(4-(bis(2-hydroxydodecyl)amino)butyl) piperazine-2,5-dione (“cKK-E12”), (((3,6-dioxopiperazine-2,5-diyl)bis(butane-4, 1-diyl))bis(azanetriyl))tetrakis(ethane-2,1-diyl) (9Z,9′Z,9″Z,9″Z,12Z,12′Z,12″Z,12″Z)-tetrakis(octadeca-9,12-dienoate) (“OF-Deg-Lin”), tetrakis(8-methylnonyl) 3,3′,3″,3″-(((methylazanediyl)bis(propane-3,1 diyl))bis (azanetriyl))tetrapropionate (“306Oᵢ₁₀”), N¹,N³,N⁵-tris(3-(didodecylamino) propyl)benzene-1,3,5-tricarboxamide (“TT3”), hexa (octan-3-yl) 9,9′,9″,9′″,9″″,9′″″-((((benzene-1,3,5-tricarbonyl)yris(azanediyl)) tris (propane-3,1-diyl)) tris(azanetriyl)) hexanonanoate (“FTT5”), or a combination thereof.

In some embodiments, the nanoparticles further comprise one or more other lipids. The one or more other lipids may include cholesterol, DC-Cholesterol, BHEM-Cholesterol, distearoylphosphatidylcholine (“DSPC”), 1,2-dimyristoyl-rac-glycero-3-methoxypolyethylene glycol-2000 (“DMG-PEG₂₀₀₀”), 1,2-dioleoyl-sn-glycero-3-phosphoethanolamine (“DOPE”), α-[2-(ditetradecylamino)-2-oxoethyl]-ω-methoxy-poly (oxy-1,2-ethanediyl) (“ALC-0159”), β-sitosterol, or a combination thereof.

In some embodiments, the nanoparticles have an average diameter of about 100 nm to about 700 nm. For example, the nanoparticles may have diameters of from 10 nm to 20 nm, from 20 nm to 30 nm, from 30 nm to 40 nm, from 40 nm to 50 nm, from 50 nm to 60 nm, from 60 nm to 70 nm, from 70 nm to 80 nm, from 80 nm to 90 nm, from 90 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 600 nm, from 600 nm to 700 nm.

### IV. Methods of Treatment

Provided herein is a method for treating a disease in a subject in need thereof, the method comprising administering to the subject in need thereof a nucleic acid molecule encoding neurofibromin protein or a derivative or fragment thereof, wherein the neurofibromin protein or fragment thereof has at least 80% homology with an amino acid sequence as depicted in SEQ ID NO: 4 or SEQ ID NO: 5. In some examples, the nucleic acid molecule has a sequence with at least 80% homology with SEQ ID NO: 2 or SEQ ID NO: 3.

Also provided herein is a method for treating a disease in a subject in need thereof, the method comprising administering to the subject in need thereof a polypeptide or a derivative or fragment thereof as described herein, wherein the polypeptide or fragment thereof has at least 80% homology with an amino acid sequence as depicted in SEQ ID NO: 4 or SEQ ID NO: 5.

Also provided herein is a method for treating a disease in a subject in need thereof, the method comprising administering to the subject in need thereof nanoparticles comprising a nucleic acid molecule encoding a neurofibromin protein or fragment thereof, wherein the neurofibromin protein or fragment thereof has at least 80% homology with an amino acid sequence as depicted in SEQ ID NO: 4 or SEQ ID NO: 5. In some examples, the nucleic acid molecule has a sequence with at least 80% homology with SEQ ID NO: 2 or SEQ ID NO: 3.

Also provided herein is a method for treating a disease in a subject in need thereof, the method comprising administering to the subject in need thereof a therapeutically effective amount of a composition comprising nanoparticles comprising a nucleic acid molecule encoding a neurofibromin protein or fragment thereof, wherein the neurofibromin protein or fragment thereof has at least 80% homology with amino acid sequence as depicted in SEQ ID NO: 4 or SEQ ID NO: 5; and a cationic lipid complex. In some examples, the nucleic acid molecule has a nucleotide sequence with at least 80% homology with SEQ ID NO: 2 or SEQ ID NO: 3.

In some embodiments, the subject in need thereof in the methods described herein is a human subject, and wherein the disease is selected from the group consisting of neurofibromatosis type 1, Relaxin, PD-L1 Autoimmune hepatitis, Nieman Pickman Disease Type C 1 (NPC1), VEGF-A Myocardial ischemia, PCCA/PCCB Propionic acidemia (PA), MUT Methylmalonic acidemia (MMA), G6Pase Glycogen Storage Disease Type 1a (GSD1a), Ornithine trans carbamylase deficiency (OTC), PAH Phenylketonuria (PKU), Crigler-Najjar Syndrome Type 1 (CN-1), and cystic fibrosis.

In some embodiments, the disease is a cancer. For example, the cancer may be selected from the group consisting of: Giloma, Sarcoma, breast cancer, endocrine cancer, melanoma, acute Imphoblastic leukemia, ovarian cancer, prostate cancer, meningioma, solid tumors/lymphoma, pancreatic cancer, and IL-12 solid tumors.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage.

By an “effective” amount (or “therapeutically effective” amount) of a composition described herein is meant a sufficient, but nontoxic amount of the agent to provide the desired effect. The term refers to an amount sufficient to treat a subject. Thus, the term therapeutic amount refers to an amount sufficient to remedy a disease state or symptoms, by preventing, hindering, retarding or reversing the progression of the disease or any other undesirable symptoms whatsoever. The term prophylactically effective amount refers to an amount given to a subject that does not yet have the disease, and thus is an amount effective to prevent, hinder or retard the onset of a disease.

The term “disease” as used herein may refer to a genetic disease in some aspects. In some aspects, the genetic disease is associated with the symptom of uncontrolled tumor growth in a subject. In some aspects, the genetic disease is neurofibromatosis type I. In some aspects, the disease is neurofibromatosis type II. The genetic disease may be Relaxin, PD-L1 Autoimmune hepatitis, Nieman Pickman Disease Type C 1 (NPC1), VEGF-A Myocardial ischemia, PCCA/PCCB Propionic acidemia (PA), MUT Methylmalonic acidemia (MMA), G6Pase Glycogen Storage Disease Type 1a (GSD1a), Ornithine trans carbamylase deficiency (OTC), PAH Phenylketonuria (PKU), Crigler-Najjar Syndrome Type 1 (CN-1), or cystic fibrosis.

The term “cancer” as used herein may refer to Glioma (low- and high-grade), Sarcoma, Breast cancer, Endocrine cancers (including pheochromocytoma and neuroendocrine tumors), Melanoma, Acute lymphoblastic leukemia, Ovarian cancer, Prostate cancer, Meningioma, Solid tumors/lymphoma, pancreatic cancer, or IL-12 Solid tumors.

The term “administering to the subject” as referred to herein includes administering to the subject a composition as described herein, by, for example, oral (including, for example, buccal or sublingual), topical, parenteral, rectal or vaginal routes, or by inhalation or spray. The term “parenteral” as used herein includes injection or infusion by subcutaneous, intradermal, intra-articular, intravenous, intramuscular, intravascular, intrasternal, intratumoral, or intrathecal routes.

In some embodiments, the subject is a mammal. In some embodiments, the subject is a human.

Toxicity and therapeutic efficacy of the formulations, agents, and compositions described herein can be determined by standard pharmaceutical procedures, using either cells in culture or experimental animals to determine the LD₅₀ (the dose lethal to 50% of the population) and the ED₅₀ (the dose therapeutically effective in 50% of the population). The dose ratio between toxic and therapeutic effects is the therapeutic index and can be expressed as the ratio LD₅₀/ED₅₀.

Data obtained from cell culture assays and further animal studies can be used in formulating a range of dosage for use in humans. The dosage of such compounds lies preferably within a range of circulating concentrations that include the ED₅₀ with little or no toxicity, and with little or no adverse effect on human health. The dosage may vary within this range depending upon the dosage form employed and the route of administration utilized. For any agents used in the methods described herein, the therapeutically effective dose can be estimated initially from cell culture assays. A dose can be formulated in animal models to achieve a circulating plasma concentration range that includes the IC₅₀ (that is, the concentration of the test compound which achieves a half-maximal inhibition of symptoms) as determined in cell culture. Such information can be used to more accurately determine useful doses in humans. Exemplary dosage amounts of the disclosed formulations, agents, or compositions are at least from about 0.01 to 3000 mg per day, e.g., at least about 0.00001, 0.0001, 0.001, 0.01, 0.1, 1, 2, 5, 10, 25, 50, 100, 200, 500, 1000, 2000, or 3000 mg per kg per day, or more.

The formulations and routes of administration can be tailored to the disease or disorder being treated, and for the specific human being treated. For example, a subject can receive a dose of the agent once or twice or more daily for one week, one month, six months, one year, or more. The treatment can continue indefinitely, such as throughout the lifetime of the human. Treatment can be administered at regular or irregular intervals (once every other day or twice per week), and the dosage and timing of the administration can be adjusted throughout the course of the treatment. The dosage can remain constant over the course of the treatment regimen, or it can be decreased or increased over the course of the treatment.

In various aspects, the dosage facilitates an intended purpose for both prophylaxis and treatment without undesirable side effects, such as toxicity, irritation or allergic response. Although individual needs may vary, the determination of optimal ranges for effective amounts of formulations is within the skill of the art. Human doses can readily be extrapolated from animal studies (Katocs et al., (1990) Chapter 27 in Remington's Pharmaceutical Sciences, 18th Ed., Gennaro, ed., Mack Publishing Co., Easton, PA). In general, the dosage required to provide an effective amount of a formulation, which can be adjusted by one skilled in the art, will vary depending on several factors, including the age, health, physical condition, weight, type and extent of the disease or disorder of the recipient, frequency of treatment, the nature of concurrent therapy, if required, and the nature and scope of the desired effect(s) (Nies et al., (1996) Chapter 3, In: Goodman & Gilman's The Pharmacological Basis of Therapeutics, 9th Ed., Hardman et al., eds., McGraw-Hill, New York, NY).

### V. Methods of Making Nanoparticle Compositions

Also provided herein are methods comprising preparing an emulsion comprising water, a polymer or a non-polymeric excipient, a solvent, and a nucleic acid molecule encoding a neurofibromin protein or fragment thereof, wherein the neurofibromin protein or fragment thereof has at least 80% homology with an amino acid sequence as depicted in SEQ ID NO: 4 or SEQ ID NO: 5; printing the emulsion using an extrusion-based printing method to generate a plurality of droplets including particles having diameters of from 10 nm to 1100 μm and comprising the polymer or the non-polymeric excipient and the nucleic acid molecule; and collecting the plurality of droplets. In some embodiments, the nucleic acid molecule has at least 80% sequence identity with SEQ ID NO: 2. In some embodiments, the nucleic acid molecule has at least 80% sequence identity with SEQ ID NO: 3.

Nanoparticles in the size range of 10-200 nm may be suitable for biomedical applications that require local or systemic administration, interaction with diseased tissues at the cellular and molecular level, and uptake into cells. The high surface area and chemical versatility of these nanoparticles may enable surface functionalization, with targeting ligands that can enhance transport across physiological barriers and provide specificity toward molecular targets characteristic of diseased tissues. At the same time, because of their macromolecular size, nanoparticles can readily act as carriers for controlled delivery of therapeutic agents, contrast agents or other cargo. As such, nanoparticles may be useful for diagnostic, therapeutic and theranostic technologies. Nanoparticles can also be incorporated into biomedical device coatings or blended as nanocomposites for the preparation of drug eluting stents, tissue engineered scaffolds, or antibacterial coatings that require the controlled release of active agents, high porosity, or nano-scaled topologies.

Compared to conventional emulsion evaporation methods, the techniques described herein are suitable for encapsulating heat-sensitive drugs, biomolecules, and live microorganisms such as bacteria, yeasts, etc. For example, by using the shear force exerted by a syringe nozzle rather than sonication energy, micro- and/or nano-sized emulsion droplets can be created, eliminating the emulsion cooling step. Furthermore, the shear force provided can be consistent and controllable, as its intensity may depend on the syringe nozzle size, printing speed, and printing pressure, which can be carefully controlled during the extrusion-based printing process.

Another advantage of the methods, systems, compositions, and techniques described herein is the ability to incorporate live cells and bacteria during the particle formulation process. Aspects described herein can combine bioprinting with particulate-based drug delivery systems in a ‘one-step’ process, useful for a variety of applications and drug delivery for different disease treatments, including particulate-based drug delivery in stem cell therapy.

In an aspect, methods are described herein, such as methods for preparing particles (e.g., microparticles and/or nanoparticles). In some examples, a method of this aspect comprises preparing an emulsion comprising water, a polymer or a non-polymeric excipient, a solvent, and an active pharmaceutical ingredient; printing the emulsion using an extrusion-based printing method to generate a plurality of droplets including particles having diameters of from 10 nm to 1000 μm and comprising the polymer or the non-polymeric excipient and the active pharmaceutical ingredient; and collecting the plurality of droplets. In some examples, the extrusion-based printing method subjects the emulsion to shear forces that separate the emulsion into the plurality of droplets including particles. Optionally the particles prepared according to this aspect may be subjected to further processing. For example, methods of this aspect may further comprise subjecting the droplets to evaporation conditions to evaporate from the droplets and leave the particles. Methods of this aspect may further comprise washing the plurality of particles, for example. Methods of this aspect may further comprise lyophilizing the plurality of droplets or the particles. In some examples, the particles may have diameters of from about 10 nm to about 1 mm or larger. For example, the particles may have diameters of from 10 nm to 20 nm, from 20 nm to 30 nm, from 30 nm to 40 nm, from 40 nm to 50 nm, from 50 nm to 60 nm, from 60 nm to 70 nm, from 70 nm to 80 nm, from 80 nm to 90 nm, from 90 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 600 nm, from 600 nm to 700 nm, from 700 nm to 800 nm, from 800 nm to 900 nm, from 900 nm to 1 μm, from 1 μm to 2 μm, from 2 μm to 3 μm, from 3 μm to 4 μm, from 4 μm to 5 μm, from 5 μm to 6 μm, from 6 μm to 7 μm, from 7 μm to 8 μm, from 8 μm to 9 μm, from 9 μm to 10 μm, from 10 μm to 20 μm, from 20 μm to 30 μm, from 30 μm to 40 μm, from 40 μm to 50 μm, from 50 μm to 60 μm, from 60 μm to 70 μm, from 70 μm to 80 μm, from 80 μm to 90 μm, from 90 μm to 100 μm, from 100 μm to 150 μm, from 150 μm to 200 μm, from 200 μm to 250 μm, from 250 μm to 300 μm, from 300 μm to 350 μm, from 350 μm to 400 μm, from 400 μm to 450 μm, from 450 μm to 500 μm, from 500 μm to 600 μm, from 600 μm to 700 μm, from 700 μm to 800 μm, from 800 μm to 900 μm, or from 900 μm to 1 mm.

A variety of different emulsions may be used for preparing particles (e.g., microparticles and/or nanoparticles) according to the aspects described herein. For example, the emulsion may comprise a water-in-oil emulsion, an oil-in-water emulsion, or a water-in-oil-in-water emulsion. Preparing the emulsion may comprise preparing a water-in-oil emulsion, an oil-in-water emulsion, or a water-in-oil-in-water emulsion. In a specific example, preparing the emulsion comprises preparing a primary emulsion comprising a water-in-oil emulsion or an oil-in-water emulsion, and preparing a secondary emulsion comprising a water-in-oil-in-water emulsion. Other components may be included in the emulsion. For example, the emulsion may comprise or further comprise one or more of a cosolvent, a surfactant, a preservative, live cells, cellular components, an additional active ingredient, a salt, a preservative, a protein, a peptide, a lipid, an amino acid, or a nucleic acid component.

The methods described herein may generally be useful for preparing particles (e.g., microparticles and/or nanoparticles) containing any desirable active ingredient. Without limitation, example active ingredients may comprise a polypeptide, an antibody, a nucleic acid, messenger ribonucleic acid (mRNA) molecules, a lipid nanoparticle, clustered regularly interspaced short palindromic repeats (CRISPR)-associated protein 9 (Cas9), transcription activator-like effector nucleases (TALENs), zinc-finger nucleases (ZFNs), homing endonucleases or meganucleases, a growth factor, a plasmid, a hydrophilic pharmaceutical, a lipophilic pharmaceutical, a viral particle, a virus-like particle, a live yeast cell, a live recombinant yeast cell, a live fungus, a live bacterial cell, a live recombinant bacterial cell, a live insect cell, a live mammalian cell, or a live mesenchymal stem cell. In some examples, a weight ratio of the active pharmaceutical ingredient to the polymer or the non-polymeric excipient in the emulsion is from 1:8 to 1:15, such as from 1:8 to 1:9, from 1:9 to 1:10, from 1:10 to 1:11, from 1:11 to 1:12, from 1:12 to 1:13, from 1:13 to 1:14, or from 1:14 to 1:15.

For particles comprising a polymer, the polymer may be a biocompatible polymer, a biodegradable polymer, or any pharmaceutically acceptable polymer. Example biodegradable polymers include, but are not limited to, poly(lactide-co-glycolide) (PLGA), polylactide (PLA), polyglycolide (PGA), polycaprolactone (PCL), pluronic F127, sodium alginate, hyaluronic acid, chitosan, cyclodextrin, dextran, agarose, gelatin, albumin, collagen, lipids, a polyethylene glycol (PEG) derivative, a pharmaceutical grade polymer, poly(hydroxy butyrate) (PHB), poly(β-malic acid) (PMA), or poly(L-lysine) (PLL).

For particles comprising a non-polymeric excipient, the non-polymeric excipient may be a hydrophilic substance or a hydrophobic substance. Example non-polymeric excipients include, but are not limited to, a non-reducing sugar, such as trehalose, or sucrose, a polyol, such as mannitol, sorbitol, xylitol, or an amino acid, such as leucine, or L-arginine.

The particles (e.g., microparticles and/or nanoparticles) can be prepared using any suitable printing parameters and any suitable environmental parameters. In some examples, the printing may occur at ambient conditions (e.g., at atmospheric pressure and at room temperature). Temperatures for collecting the plurality of droplets may correspond to ambient temperature or cryogenic temperatures. For example, collecting the plurality of droplets comprises receiving the plurality of droplets on a surface having a temperature of from about −200° C. to about −78° C. or at room temperature or from about 4° C. to about 50° C. In some examples, the extrusion-based printing method subjects the emulsion to a pressure of from 10 kPa to 700 kPa, such as from 10 kPa to 600 kPa, from 10 kPa to 500 kPa, from 10 kPa to 400 kPa, from 10 kPa to 300 kPa, from 10 kPa to 200 kPa, from 10 kPa to 100 kPa, from 10 kPa to 20 kPa, from 20 kPa to 30 kPa, from 30 kPa to 40 kPa, from 40 kPa to 50 kPa, from 50 kPa to 60 kPa, from 60 kPa to 70 kPa, from 70 kPa to 80 kPa, from 80 kPa to 90 kPa, from 90 kPa to 100 kPa, from 110 kPa to 120 kPa, from 120 kPa to 130 kPa, from 130 kPa to 140 kPa, from 140 kPa to 150 kPa, from 150 kPa to 160 kPa, from 160 kPa to 170 kPa, from 170 kPa to 180 kPa, from 180 kPa to 190 kPa, from 190 kPa to 200 kPa, from 200 kPa to 210 kPa, from 210 kPa to 220 kPa, from 220 kPa to 230 kPa, from 230 kPa to 240 kPa, from 240 kPa to 250 kPa, from 250 kPa to 300 kPa, from 300 kPa to 350 kPa, from 350 kPa to 400 kPa, from 400 kPa to 450 kPa, from 450 kPa to 500 kPa, from 500 kPa to 550 kPa, from 550 kPa to 600 kPa, from 600 kPa to 650 kPa, or from 650 kPa to 700 kPa. Optionally, an extrusion pressure of the extrusion-based printing method greater than or about 700 kPa. In some examples, the extrusion-based printing method uses a nozzle having a diameter of from 1 μm to 1000 μm, such as from 1 μm to 10 μm, from 10 μm to 100 μm, from 100 μm to 700 μm, from 300 μm to 700 μm, from 100 μm to 200 μm, from 200 μm to 300 μm, from 300 μm to 400 μm, from 400 μm to 500 μm, from 500 μm to 600 μm, from 600 μm to 700 μm, from 700 μm to 800 μm, from 800 μm to 900 μm, or from 900 μm to 1000 μm. Optionally, a temperature of the emulsion during the printing is from about 4° C. to about 50° C., such as from 4° C. to 10° C., from 10° C. to 20° C., from 20° C. to 30° C., from 30° C. to 40° C., or from 40° C. to 50° C. Optionally, printing the emulsion comprises receiving the particles on a surface, wherein the surface has a temperature of about room temperature or less than or about −180° C.

In another aspect, systems are described herein, such as systems for preparing particles (e.g., microparticles and/or nanoparticles), optionally according to the methods described herein. In some examples, a system of this aspect comprises an emulsion supply container for preparing or storing an emulsion; one or more extrusion-based printing nozzles in fluid communication with the emulsion supply container for generating a plurality of droplets of the emulsion including particles, such as having diameters of from 10 nm to 1000 μm; and a collection surface for receiving the plurality of droplets of the emulsion from the one or more extrusion-based printing nozzles. In examples, the emulsion may comprise water, a polymer or a non-polymeric excipient, a solvent, and an active pharmaceutical ingredient. In some examples, the collection surface comprises a sterile vial.

Systems of this aspect can include various components or adjustable parameters to allow for preparing particles (e.g., microparticles and/or nanoparticles), such as according to the methods described herein. For example, systems of this aspect may further comprise one or more mixing vessels in fluid communication with the emulsion supply container for preparing and providing the emulsion to the emulsion supply container. In some examples, the collection surface may optionally be cooled to a temperature of from about −200° C. to about −75° C. A system of this aspect may further comprise a cooling or refrigeration system coupled to the collection surface for cooling the collection surface to a temperature of from about −200° C. to about −75° C. Optionally, a system of this aspect may comprise one or more temperature sensors or temperature controllers for monitoring or controlling a temperature of the collection surface. In some examples, the collection surface is a moving or movable or translating or translatable collection surface. Optionally, a system of this aspect may further comprise a translation stage for generating a relative translation between the one or more extrusion-based printing nozzles and the collection surface. In some examples, a system of this aspect may further comprise one or more pressure sensors or pressure controllers for monitoring or controlling an extrusion pressure associated with the one or more extrusion-based printing nozzles. Optionally, a system of this aspect may further comprise one or more actuators for monitoring or controlling an extrusion speed associated with the one or more extrusion-based printing nozzles.

In some examples, it may be desirable to prepare particles (e.g., microparticles and/or nanoparticles) under sterile conditions. Optionally, a system of this aspect may further comprise a housing for maintaining at least the one or more extrusion-based printing nozzles and the collection surface in a sterile environment. Optionally, a system of this aspect may further comprise sterilization equipment positioned to sterilize one or more of the emulsion supply container, the one or more extrusion-based printing nozzles, or the collection surface.

In another aspect compositions are provided herein, such as microparticle-based therapeutic compositions. In some examples, a composition may comprise particles having diameters of from 10 nm to 1000 μm; and one or more live cells. In some examples, the particles may have diameters of from about 10 nm to about 1 mm or larger. For example, the particles may have diameters of from 10 nm to 20 nm, from 20 nm to 30 nm, from 30 nm to 40 nm, from 40 nm to 50 nm, from 50 nm to 60 nm, from 60 nm to 70 nm, from 70 nm to 80 nm, from 80 nm to 90 nm, from 90 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 600 nm, from 600 nm to 700 nm, from 700 nm to 800 nm, from 800 nm to 900 nm, from 900 nm to 1 μm, from 1 μm to 2 μm, from 2 μm to 3 μm, from 3 μm to 4 μm, from 4 μm to 5 μm, from 5 μm to 6 μm, from 6 μm to 7 μm, from 7 μm to 8 μm, from 8 μm to 9 μm, from 9 μm to 10 μm, from 10 μm to 20 μm, from 20 μm to 30 μm, from 30 μm to 40 μm, from 40 μm to 50 μm, from 50 μm to 60 μm, from 60 μm to 70 μm, from 70 μm to 80 μm, from 80 μm to 90 μm, from 90 μm to 100 μm, from 100 μm to 150 μm, from 150 μm to 200 μm, from 200 μm to 250 μm, from 250 μm to 300 μm, from 300 μm to 350 μm, from 350 μm to 400 μm, from 400 μm to 450 μm, from 450 μm to 500 μm, from 500 μm to 600 μm, from 600 μm to 700 μm, from 700 μm to 800 μm, from 800 μm to 900 μm, or from 900 μm to 1 mm. In some examples, the particles (e.g., microparticles and/or nanoparticles) are attached to surfaces of the one or more live cells. In some examples, the one or more live cells are at least partially encapsulated into the particles. Example live cells include, but are not limited to, live yeast cells, live recombinant yeast cells, live fungal cells, live bacterial cells, live recombinant bacterial cells, live insect cells, live mammalian cells, or live mesenchymal stem cells. Optionally, the particles may be in a lyophilized condition.

Example particles include microparticles or nanoparticles comprising a polymer or a non-polymeric excipient, such as prepared according to various methods described herein or prepared using various systems described herein. Optionally, for particles comprising a polymer, the polymer is a biodegradable polymer selected from the group consisting of poly(lactide-co-glycolide), polylactide (PLA), polyglycolide (PGA), polycaprolactone (PCL), pluronic F127, sodium alginate, hyaluronic acid, chitosan, cyclodextrin, dextran, agarose, gelatin, albumin, collagen, lipids, a polyethylene glycol (PEG) derivative, a pharmaceutical grade polymer, poly(hydroxy butyrate) (PHB), poly(β-malic acid) (PMA), or poly(L-lysine) (PLL). Optionally, for particles comprising a non-polymeric excipient, the non-polymeric excipient is a hydrophilic substance, a hydrophobic substance, a non-reducing sugar, trehalose, sucrose, a polyol, mannitol, sorbitol, xylitol, an amino acid, leucine, or L-arginine.

In some examples, microparticles and nanoparticles can be prepared using the printheads described herein. For example, the printheads can be used for extrusion-based printing and emulsion/mixture evaporation techniques to fabricate novel polymeric microparticles or nanoparticles, such as comprising polymeric poly(lactide-co-glycolide) (PLGA) or other materials, which may be optionally biocompatible or biodegradable. PLGA is an example biocompatible and biodegradable FDA-approved copolymer, which can be hydrolyzed into lactic and glycolic acid monomers.

In an aspect, methods are described herein, such as methods for preparing particles (e.g., microparticles and/or nanoparticles). In some examples, a method of this aspect comprises providing a first component to a first linker of a plurality of linkers of a printhead; providing a second component to a second linker of the plurality of linkers of the printhead; combining the first component and the second component in a fluid mixer of the printhead;

forming a mixture of the first component and the second component in the printhead; and

printing or flowing the mixture from the printhead through one or more outlets. In some examples, the first component comprises a polymer, a non-polymeric excipient, or an active pharmaceutical ingredient. In some examples, the second component comprises a solvent. In some examples, additional components can be provided to additional linkers of the plurality of linkers, such that the additional components are mixed with the first and second component in the fluid mixer. Printing may subject the mixture to shear forces that separate the mixture into a plurality of droplets including particles.

Combining the first component and the second component may comprise providing the first component to an inlet of a fluid mixer in the printhead and providing the second component to the same or a different inlet of the fluid mixer in the printhead. The fluid mixer may comprise a coaxial or other arrangement of a first fluidic channel in fluid communication with a first linker and a second fluid channel in fluid communication with a second linker, for example. Optionally, the first component is provided to the first linker, and the second component is provided to the second linker, which can direct the components to the fluid mixer. In some examples, the fluid mixer may comprise a stirrer. Optionally, an electric field or a magnetic field may be used for activating the stirrer. The stirrer may optionally rotate or be rotatable about an axis parallel to a flow of the mixture (e.g., through one or more of the outlets) or one or more components, or about an axis perpendicular to a flow of the mixture or one or more of the components, or about an axis with any other orientation relative to the flow of the mixture or one or more of the components. The fluid mixer may optionally comprise a mixing architecture characterized by an “S” shape, a “Y” shape, or a helix shape. Again, one or more additional linkers can be included in the printhead to allow for mixing of 3 or more components in the fluid mixer.

Optionally, the particles and/or droplets prepared according to this aspect may be subjected to further processing. For example, methods of this aspect may further comprise subjecting the droplets to evaporation conditions to evaporate the solvent from the droplets and leave the particles. Methods of this aspect may further comprise washing the plurality of particles, for example. Methods of this aspect may further comprise lyophilizing the plurality of droplets or the particles.

Other components may be included in the mixture. For example, the mixture may comprise or further comprise one or more of a cosolvent, a surfactant, a preservative, live cells, cellular components, an additional active ingredient, a salt, a preservative, a polypeptide, a peptide, an amino acid, or a nucleic acid component.

The methods described herein may generally be useful for preparing particles containing any desirable active ingredient. Without limitation, example active ingredients may comprise a polypeptide, an antibody, a nucleic acid molecule, messenger ribonucleic acid (mRNA) molecules, a lipid nanoparticle, clustered regularly interspaced short palindromic repeats (CRISPR)-associated protein 9 (Cas9), transcription activator-like effector nucleases (TALENs), zinc-finger nucleases (ZFNs), homing endonucleases or mega-nucleases, a growth factor, a plasmid, a hydrophilic pharmaceutical, a lipophilic pharmaceutical, a viral particle, a virus-like particle, a live yeast cell, a live recombinant yeast cell, a live fungus, a live bacterial cell, a live recombinant bacterial cell, a live insect cell, a live mammalian cell, or a live mesenchymal stem cell. In some examples, a weight ratio of the active pharmaceutical ingredient to the polymer or the non-polymeric excipient in the mixture is from 1:8 to 1:15, such as from 1:8 to 1:9, from 1:9 to 1:10, from 1:10 to 1:11, from 1:11 to 1:12, from 1:12 to 1:13, from 1:13 to 1:14, or from 1:14 to 1:15.

For particles comprising a polymer, the polymer may be a biodegradable polymer. Example biodegradable polymers include, but are not limited to, poly(lactide-co-glycolide), polylactide (PLA), polyglycolide (PGA), polycaprolactone (PCL), Pluronic F127, sodium alginate, hyaluronic acid, chitosan, cyclodextrin, dextran, agarose, gelatin, albumin, collagen, lipids, a polyethylene glycol (PEG) derivative, a pharmaceutical grade polymer, poly(hydroxy butyrate) (PHB), poly(β-malic acid) (PMA), or poly(L-lysine) (PLL).

For particles comprising a non-polymeric excipient, the non-polymeric excipient may be a hydrophilic substance or a hydrophobic substance. Example non-polymeric excipients include, but are not limited to, a non-reducing sugar, such as trehalose, or sucrose, a polyol, such as mannitol, sorbitol, xylitol, or an amino acid, such as leucine, or L-arginine.

The particles can be prepared using any suitable printing parameters and any suitable environmental parameters. In some examples, the printing may occur at ambient conditions (e.g., at atmospheric pressure and at room temperature), though control of the temperature of the mixture may be achieved by including a heat exchanger or other temperature controller (e.g., a coolant jacket, a Peltier cooler, etc.) in the printhead (e.g., at the fluid mixer). Temperatures for collecting the plurality of droplets may correspond to ambient temperature or cryogenic temperatures. For example, collecting the plurality of droplets optionally comprises receiving the plurality of droplets on a surface having a temperature of from about −200° C. to about −78° C. or at room temperature or from about 4° C. to about 50° C. In some examples, the extrusion-based printing method subjects the mixture to a pressure of from 1 kPa to 700 kPa, such as from 1 kPa to 600 kPA, from 1 kPa to 500 kPa, from 1 kPa to 400 kPa, from 1 kPa to 300 kPa, from 1 kPa to 200 kPa, from 1 kPa to 100 kPa, from 1 kPa to 50 kPa, from 1 kPa to 40 kPa, from 1 kPa to 30 kPa, from 1 kPa to 20 kPa, from 1 kPa to 10 kPa, from 10 kPa to 600 kPa, from 10 kPa to 500 kPa, from 10 kPa to 400 kPa, from 10 kPa to 300 kPa, from 10 kPa to 200 kPa, from 10 kPa to 100 kPa, from 10 kPa to 20 kPa, from 20 kPa to 30 kPa, from 30 kPa to 40 kPa, from 40 kPa to 50 kPa, from 50 kPa to 60 kPa, from 60 kPa to 70 kPa, from 70 kPa to 80 kPa, from 80 kPa to 90 kPa, from 90 kPa to 100 kPa, from 110 kPa to 120 kPa, from 120 kPa to 130 kPa, from 130 kPa to 140 kPa, from 140 kPa to 150 kPa, from 150 kPa to 160 kPa, from 160 kPa to 170 kPa, from 170 kPa to 180 kPa, from 180 kPa to 190 kPa, from 190 kPa to 200 kPa, from 200 kPa to 210 kPa, from 210 kPa to 220 kPa, from 220 kPa to 230 kPa, from 230 kPa to 240 kPa, from 240 kPa to 250 kPa, from 250 kPa to 300 kPa, from 300 kPa to 350 kPa, from 350 kPa to 400 kPa, from 400 kPa to 450 kPa, from 450 kPa to 500 kPa, from 500 kPa to 550 kPa, from 550 kPa to 600 kPa, from 600 kPa to 650 kPa, or from 650 kPa to 700 kPa. Optionally, an extrusion pressure of the extrusion-based printing method greater than or about 700 kPa. In some examples, the extrusion-based printing method uses a nozzle having a diameter of from 1 μm to 1000 μm, such as from 1 μm to 10 μm, from 10 μm to 100 μm, from 100 μm to 700 μm, from 300μ m to 700 μm, from 100μ m to 200 μm, from 200μ m to 300 μm, from 300 μm to 400 μm, from 400 μm to 500 μm, from 500 μm to 600 μm, from 600 μm to 700 μm, from 700 μm to 800 μm, from 800 μm to 900 μm, or from 900 μm to 1000 μm. Optionally, a temperature of the mixture during the printing is from about 4° C. to about 50° C., such as from 4° C. to 10° C., from 10° C. to 20° C., from 20° C. to 30° C., from 30° C. to 40° C., or from 40° C. to 50° C. Optionally, printing the mixture comprises receiving the particles or droplets on a surface, wherein the surface has a temperature of about room temperature or less than or about −180° C.

In another aspect, systems are described herein, such as systems for preparing particles (e.g., microparticles and/or nanoparticles), optionally according to the methods described herein. Optionally, a system of this aspect comprises a printer, such as a bioprinter, which may include one or more pneumatically controlled fluid extruders. In some examples, systems of this aspect comprise a plurality of supply containers for preparing or storing respective components, such as but not limited to components comprising a polymer or a non-polymeric excipient, a solvent, or an active pharmaceutical ingredient; a printhead in fluid communication with the plurality of supply containers, the printhead comprising, a plurality of linkers, such as where each linker is in fluid communication with an outlet of one of the plurality of supply containers; a fluid mixer for mixing components from the plurality of linkers into a mixture; and one or more outlets in fluid communication with the fluid mixer. The system may optionally comprise a collection surface for receiving a plurality of droplets of the mixture from the one or more outlets. In examples, the mixture may comprise water, a polymer or a non-polymeric excipient, a solvent, and an active pharmaceutical ingredient. In some examples, the collection surface comprises a sterile vial.

Systems of this aspect can include various components or adjustable parameters to allow for preparing particles, such as according to the methods described herein. In some examples, the collection surface may optionally be cooled to a temperature of from about −200° C. to about −75° C. A system of this aspect may further comprise a cooling or refrigeration system coupled to the collection surface for cooling the collection surface to a temperature of from about −200° C. to about −75° C. Optionally, a system of this aspect may comprise one or more temperature sensors or temperature controllers for monitoring or controlling a temperature of the collection surface. In some examples, the collection surface is a moving or movable or translating or translatable collection surface. Optionally, a system of this aspect may further comprise a translation stage for generating a relative translation between one or more printheads and/or nozzles and the collection surface. In some examples, a system of this aspect may further comprise one or more pressure sensors or pressure controllers for monitoring or controlling an extrusion pressure associated with the printheads and/or nozzles. Optionally, a system of this aspect may further comprise one or more actuators for monitoring or controlling an extrusion speed associated with one or more printheads and/or nozzles.

In some examples, it may be desirable to prepare particles under sterile conditions. Optionally, a system of this aspect may further comprise a housing for maintaining at least one or more printheads and/or nozzles and the collection surface in a sterile environment. Optionally, a system of this aspect may further comprise sterilization equipment positioned to sterilize one or more of the plurality of supply containers, the one or more printheads and/or nozzles, or the collection surface.

In another aspect compositions are provided herein, such as microparticle-based therapeutic compositions. In some examples, a composition may comprise particles having diameters of from 10 nm to 1100 μm; and one or more live cells. In some examples, the particles are attached to surfaces of the one or more live cells. In some examples, the one or more live cells are at least partially encapsulated into the particles. Example live cells include, but are not limited to, live yeast cells, live recombinant yeast cells, live fungal cells, live bacterial cells, live recombinant bacterial cells, live insect cells, live mammalian cells, or live mesenchymal stem cells. Optionally, the particles may be in a lyophilized condition.

Example particles include particles comprising a polymer or a non-polymeric excipient, such as prepared according to various methods described herein or prepared using various systems described herein. Optionally, for particles comprising a polymer, the polymer is a biodegradable polymer selected from the group consisting of poly(lactide-co-glycolide), polylactide (PLA), polyglycolide (PGA), polycaprolactone (PCL), pluronic F127, sodium alginate, hyaluronic acid, chitosan, cyclodextrin, dextran, agarose, gelatin, albumin, collagen, lipids, a polyethylene glycol (PEG) derivative, a pharmaceutical grade polymer, poly(hydroxy butyrate), poly(β-malic acid), or poly(L-lysine). Optionally, for particles comprising a non-polymeric excipient, the non-polymeric excipient is a hydrophilic substance, a hydrophobic substance, a non-reducing sugar, trehalose, sucrose, a polyol, mannitol, sorbitol, xylitol, an amino acid, leucine, or L-arginine.

In some examples, the particles further comprise an active ingredient embedded within or adsorbed to the particles. For example, the active pharmaceutical ingredient may include one or more of a polypeptide, an antibody, a nucleic acid, messenger ribonucleic acid (mRNA) molecules, a lipid nanoparticle, clustered regularly interspaced short palindromic repeats (CRISPR)-associated protein 9 (Cas9), transcription activator-like effector nucleases (TALENs), zinc-finger nucleases (ZFNs), homing endonucleases or meganucleases, a growth factor, a plasmid, a hydrophilic pharmaceutical, a lipophilic pharmaceutical, a viral particle, a virus-like particle, a live yeast cell, a live recombinant yeast cell, a live fungus, a live bacterial cell, a live recombinant bacterial cell, a live insect cell, a live mammalian cell, or a live mesenchymal stem cell.

In another aspect, printheads are described herein, such as printheads for preparing particles, optionally according to the methods described herein. In some examples, the printheads may comprise or correspond to various printheads described herein. The printheads may include a plurality of linkers defining inlets of the printhead; a fluid mixer in fluid communication with the plurality of linkers for mixing components from the inlets into a mixture; and one or more outlets in fluid communication with the fluid mixer.

Optionally, one or more of the plurality of linkers comprises a Luer lock, a Luer slip, or a slip tip. In some embodiments, the plurality of linkers may be characterized by a diameter of less than or about 5.0 mm. The linkers may be used and/or configured for establishing sealed fluid communication with other devices, such as a bioprinter, pneumatically controlled fluid extruders, supply containers, other printheads or fluid mixers, etc.

In some examples, the fluid mixer may comprise a coaxial or other arrangement of a first fluidic channel in fluid communication with a first linker and a second fluid channel in fluid communication with a second linker. One or more additional fluidic channels in fluid communication with one or more additional linkers may optionally be used. In some embodiments, the fluid mixer may comprise a stirrer, which may or may not include an electric field or a magnetic field for activating the stirrer. The stirrer may optionally rotate or be rotatable about an axis parallel to a flow of the mixture (e.g., through one or more of the outlets) or one or more components, or about an axis perpendicular to a flow of the mixture or one or more of the components, or about an axis with any other orientation relative to the flow of the mixture or one or more of the components. The fluid mixer may be formed from a simple 2D structure to a complex 2D structure to a 3D structure. The fluid mixer may be any shape with any angle size/channel cross-section and, in embodiments, may comprise a mixing architecture characterized by a “Y” shape, an “S” shape, or a helix shape. In some cases, mixing architecture can be configured according to any suitable shape, configuration, or scheme for a particular application, such as to achieve a specific amount of mixing and/or to achieve complete and/or partial mixing of two or three or more different components, for example.

In some examples, the one or more outlets may be characterized by a diameter of less than or about 5.0 mm. The one or more outlets may comprise one or more extrusion-based printing nozzles for generating a plurality of droplets of the mixture including particles having diameters of from 10 nm to 1100 μm. In some examples, the one or more outlets may comprise additional linkers, such as for establishing fluid communication with a linker of an additional printhead, such as to provide a mixture of two or more components as an input for the additional printhead.

In another aspect, methods are described herein, such as methods for preparing and/or manufacturing printheads. In some examples, any of the printheads described herein can be prepared or manufactured. In some examples, the printhead comprises a plurality of linkers defining inlets of the printhead; a fluid mixer in fluid communication with the plurality of linkers for mixing components from the inlets into a mixture; and one or more outlets in fluid communication with the fluid mixer.

In some examples, multiple printheads may be linked together, such as where a linker of one printhead is coupled to an outlet of another printhead. Such a configuration may be useful for achieving complex mixing configurations, such as where a coaxial or multi-axial mixture flow of components is achieved by a first printhead and where the mixture flow from the first printhead is further mixed with additional components, such as in a further coaxial or multi-axial mixture flow.

In some examples, manufacturing the printhead may comprise an additive manufacturing process (e.g., 3D printing using 3D laser sintering, SLA printing, or the like). The methods may include 3D modelling the printhead prior to manufacturing the printhead. The methods may include designating a 3D model for the printhead. Optionally, the method may include modeling flow and mixing within the fluid mixer prior to manufacturing the printhead.

The invention may be further understood by the following non-limiting examples.

### Example 1: Luciferase RNA Delivery

Luciferase RNA was chosen as a control RNA to evaluate and optimize lipid nanoparticles. Luciferase Control RNA was procured from Promega (USA). The RNA is a unique functional control for in vitro translation reactions. Luciferase Control RNA is an uncapped in vitro-transcribed RNA containing a 30-base poly(A) tail that produces functional luciferase when translated.

### Example 2: Preparation of NF1 mRNA

The NF1 gene encodes for the neurofibroma protein that acts as a tumor suppressor. The mutation of this gene result in progression of cancerous condition. An NF1 mRNA sequence, SEQ ID NO: 1, was analyzed and optimized to avoid the sequences of any restriction enzymes using open access tools provided by National Center for Biotechnology Information (“NCBI”). Then the modified sequences were restricted until the presence of the stop codons, where SEQ ID NO: 2 and SEQ ID NO: 3 were the resulting optimized nucleotide sequences. The sequence was used to generate the mRNA following the steps of Plasmid linearization (BbsI), Transcription, Dnase, purification of the crude RNA using Rneasy kit, Phosphatase treatment for incorporation of poly(A) tail, and a final purification with Rneasy kit. The purified mRNA can be employed for preparation of lipid-mRNA nanoparticles.

### Example 3: Erythropoeitin

Erythropoietin (EPO) was purchased from Trilink technologies. The sequence of the mRNA was developed by Trilink technologies. An mRNA sequence for EPO available in the public domain can be found in the NCBI reference sequence NM_000799.4 (herein incorporated by reference), where the encoded EPO is NCBI reference sequence NP_000790.2 (herein incorporated by reference). It will be understood by one skilled in the art that compositions and nanoparticles described herein may also be prepared using the NCBI reference sequence NM_000799.4 to encode EPO.

### Example 4: Methods

### Preparation of Lipid Nanoparticles

DSPC, cholesterol, DOTAP, and DMG-PEG₂₀₀₀ were mixed together at a molar ratio of 10:48:40:2 for DSPC:cholesterol:DOTAP:DMG-PEG₂₀₀₀. Typically, 365.8 μL DOTAP, 69.2 μL DMG-PEG₂₀₀₀, 256 μL cholesterol, and 109 μL DSPC were mixed in ethanol to get a stock solution of 6 mg/mL. The Nitrogen: phosporous ratio (N/P) of the cationic lipid:mRNA of 0.1, 1, 10, and 100 were prepared with different ratios provided in the table. The mRNA (encoding EPO in this Example) was kept constant as 1 μg. Dilute the lipid mixture as appears in Table 1.

TABLE 1

| | Lipid stock | |
|---|---|---|
| N/P | (μL) | Ethanol |
| 0.1 | 0.1 | 999.9 |
| 1 | 1.2 | 998.8 |
| 10 | 12.5 | 987 |
| 100 | 125 | 875 |

### SMART Process

The lipid nanoparticles may be prepared and optimized using Sprayed Multi Adsorbed Particle Reposing Technology (SMART) a novel additive manufacturing-based method developed in-house. The technology is a combination of extrusion-based printing and emulsion evaporation techniques. Herein, the printed mRNA particulate systems may be flash frozen (at or below the temperature of −80° C.) right at the print bed continuously and the frozen immobilized system may then be lyophilized to increase their stability and facilitate their storage and transportation in a truly integrated continuous system. The process applies the least possible shear stress eliminating any heat generation which is beneficial for formulating tension-sensitive materials such as mRNA. Thus, this technology is a safer option for processing heat sensitive molecules compared to conventional methods. Also, production yield is estimated to be almost 100% since the continuous one-step technology minimizes material waste.

### Characterization

### Particle Size

The particle size of lipid nanoparticles was measured using a Zetasizer Nano instrument from Malvern Pananalytical, following dynamic light scattering principle.

### Gel-Retardation Assay

The 1% agarose gels were prepared by mixing 0.5 g of agarose in 49 mL 1× Tris Acetate-Ethylenediaminetetraacetic acid buffer (“1×TAE buffer”) and solidified in a gel casting unit of the electrophoresis. After gelation, the gel trough was submerged in the gel tank filled with 400 mL 1×TAE buffer. The RNA marker, nanoparticles of different N/P ratios are mixed with 6× gel loading dye and 25 μL of the sample was loaded in each well. The gel was run at 75V for 60 min. The gel was imaged under UV transilluminator, and optical images were taken using a camera equipped mobile phone.

### Toxicity Assay

The osteosarcoma MG63 cells were procured from ATCC. The cells were cultured in Dulbecco's modified eagles' medium of low glucose supplemented with 10% fetal bovine serum and 1% penicillin-streptomycin in a 5% Co₂ incubator at 37° C. For the assay, 10,000 cells were added to each well in a 96 well plate for 24 hours. The lipid-mRNA complexes (containing mRNA encoding EPO) were added in different volumes ranging from 10-100 μL for 48 hours. To evaluate the cell viability, 10% 3-(4,5-Dimethylthiazol-2-yl)-2,5-Diphenyltetrazolium Bromide (“MTT reagent”) (5 mg/mL) in the cell culture media was added to the cells and incubated for 2 hours. The media was removed and 100 μL cell culture media was added to each well. The color change was measured at 570 nm and 700 nm.

### STATEMENTS REGARDING INCORPORATION BY REFERENCE AND VARIATIONS

All references throughout this application, for example, patent documents including issued or granted patents or equivalents; patent application publications; and non-patent literature documents or other source material; are hereby incorporated by reference herein in their entireties, as though individually incorporated by reference.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art.

When a group of substituents is disclosed herein, it is understood that all individual members of those groups and all subgroups and classes that can be formed using the substituents are disclosed separately. When a Markush group or other grouping is used herein, all individual members of the group and all combinations and sub-combinations possible of the group are intended to be individually included in the disclosure. As used herein, “and/or” means that one, all, or any combination of items in a list separated by “and/or” are included in the list; for example, “1, 2, and/or 3” is equivalent to “1, 2, 3, 1 and 2, 1 and 3, 2 and 3, or 1, 2 and 3”.

Every formulation or combination of components described or exemplified can be used to practice the invention unless otherwise stated. Specific names of materials are intended to be exemplary, as it is known that one of ordinary skill in the art can name the same material differently. It will be appreciated that methods, device elements, starting materials, and synthetic methods other than those specifically exemplified can be employed in the practice of the invention without resorting to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges, and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element, elements, limitation, or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art and that such modifications and variations are considered to be within the scope of this invention as defined by the appended claims.

1. A nucleic acid molecule encoding a polypeptide comprising the amino acid sequence of SEQ ID NO: 4 or SEQ ID NO:5.

2. (canceled)

3. A nucleic acid molecule comprising a modified SEQ ID NO: 1, wherein the modified SEQ ID NO: 1 comprises removal of stop codons or removal of restriction enzyme sites [GCGATG] relative to SEQ ID NO: 1.

4. The nucleic acid molecule of claim 3, wherein the nucleic acid molecule encodes a neurofibromin protein.

5. The nucleic acid molecule of claim 3, wherein the nucleic acid molecule has at least 80% or at least 90% sequence identity with SEQ ID NO: 2 or SEQ ID NO: 3.

6. (canceled)

7. A nucleic acid molecule encoding a neurofibromin protein or derivative or fragment thereof, wherein the neurofibromin protein or derivative or fragment thereof has at least 80% homology with the amino acid sequence as depicted in SEQ ID NO: 4 or SEQ ID NO: 5.

8. The nucleic acid molecule of claim 7, wherein the nucleic acid molecule has at least 90%, at least 95%, or at least 99% sequence identity with SEQ ID NO: 2 or SEQ ID NO: 3.

9-10. (canceled)

11. An expression vector comprising the nucleic acid molecule of claim 7.

12. An mRNA or fragment thereof comprising the nucleic acid molecule of claim 5.

13. (canceled)

14. A composition comprising:
the nucleic acid molecule of claim 7; and
a cationic lipid complex.

15. The composition of claim 14, further comprising a cationic lipid, an ionic lipid, or a polymer.

16-17. (canceled)

18. The composition of claim 14, wherein the composition comprises
nanoparticles comprising the nucleic acid molecule of claim 7.

19-20. (canceled)

21. The composition of claim 18, wherein the nanoparticles further comprise a cationic polymer, cholesterol, ionic lipids, or a mixture thereof.

22. The composition of claim 21, wherein the cationic polymer is DOTAP, DPSE, or a mixture thereof.

23. The composition of claim 18, wherein the nanoparticles have an average diameter of about 100 nm to about 700 nm.

24. A method for treating a disease in a subject in need thereof, the method comprising:
administering to the subject a therapeutically effective amount of a composition comprising the nucleic acid molecule of claim 7.

25. (canceled)

26. The method of claim 24, wherein the disease is a genetic disease or disorder.

27. The method of claim 24, wherein the subject is a human subject, and wherein the disease is selected from the group consisting of: neurofibromatosis type 1, Relaxin, PD-L1 Autoimmune hepatitis, Nieman Pickman Disease Type C 1 (NPC1), VEGF-A Myocardial ischemia, PCCA/PCCB Propionic acidemia (PA), MUT Methylmalonic acidemia (MMA), G6Pase Glycogen Storage Disease Type 1a (GSD1a), Ornithine transcarbamylase deficiency (OTC), PAH Phenylketonuria (PKU), Crigler-Najjar Syndrome Type 1 (CN-1), cystic fibrosis, cancer, Glioma, Sarcoma, breast cancer, endocrine cancer, melanoma, acute, lymphoblastic leukemia, ovarian cancer, prostate cancer, meningioma, solid tumors/lymphoma, pancreatic cancer, and IL-12 solid tumors.

28-29. (canceled)

30. The method of claim 24, wherein the nucleic acid molecule has at least 80% or at least 90% sequence identity with SEQ ID NO: 2 or SEQ ID NO: 3.

31. (canceled)

32. A method comprising:
preparing an emulsion comprising water, a polymer or a non-polymeric excipient, a solvent, and the nucleic acid molecule of claim 7;
printing the emulsion using an extrusion-based printing method to generate a plurality of droplets including particles having diameters of from 10 nm to 1100 μm and comprising the polymer or the non-polymeric excipient and the nucleic acid molecule; and
collecting the plurality of droplets.

33. The method of claim 32, wherein the nucleic acid molecule has at least 80% or at least 90% sequence identity with SEQ ID NO: 2 or SEQ ID NO: 3.

34. (canceled)
